(19) United States
(12) Patent Application Publication
(10) Pub. No.: US 2024/0093217 A1
(43) Pub. Date: Mar. 21, 2024

(54) RICE SUBERIN BIOSYNTHETIC GENES AND REGULATORS

(71) Applicant: The Regents of the University of California, Oakland, CA (US)

(72) Inventors: Julia Bailey-Serres, Orange, CA (US); Alexander T. Borowsky, Riverside, CA (US); Mauricio Reynoso, La Plata (AR)

(21) Appl. No.: 18/449,242

(22) Filed: Aug. 14, 2023

Related U.S. Application Data
(60) Provisional application No. 63/371,364, filed on Aug. 12, 2022.

(51) Int. Cl. C12N 15/82

(52) U.S. Cl. CPC C12N 15/8243; C12N 15/8273

(57) ABSTRACT

The present disclosure provides a list of genes, and the proteins encoded by these genes, that modulate and/or participate in the synthesis of the biopolymer suberin. The genes described here are useful in methods for producing genetically modified plants or breeding plants with increased or decreased suberin. Such plants can contain modified, mutated, or engineered candidate peptides; or have disrupted or enhanced expression using methods such as clustered regularly-interspaced short palindromic repeats (CRISPR)/CRISPR associated (Cas) nuclease, an antisense nucleic acid, a zinc finger nuclease (ZFN), or a transcription activator-like effector (TALE) nuclease. Increased suberin has a positive influence on response to plant water stress and pathogen protection, and a long-lasting role as a carbon sink in soil; and decreased suberin encourages symbioses and nutrient uptake.

### CROSS-REFERENCE TO RELATED APPLICATIONS

This application claims benefit of and priority to U.S. provisional application No. 63/371,364, filed Aug. 12, 2022, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under Grant No. #IOS-1238243, awarded by the National Science Foundation. The Government has certain rights in the invention.

### SEQUENCE LISTING

The instant application contains a Sequence Listing which has been submitted electronically in XML format and is hereby incorporated by reference in its entirety. Said XML copy, created on Oct. 6, 2023, is named 081906-1397175-248410US SL.xml and is 27,384 bytes in size.

### BACKGROUND OF THE INVENTION

Suberin is a complex carbon-rich biopolymer typically found in plant cell walls. Because suberized cell walls act as barriers that limit water and nutrient transport as well as protect plants from invasion by pathogens, suberization is increased in roots in response to drought, salt stress or oxygen deficiency. Suberin is present ubiquitously in specific internal root-tissues of vascular plants. For example, suberin is produced by the outer periderm of potatoes and tomato fruit as a barrier to moisture loss, and potato mutants that fail to deposit suberin have short shelf-lives. Nevertheless, the genetics determining suberin deposition and regulation in most plant species has been largely unknown.

Rice is notable among crops for its ability to thrive a waterlogged soils and endure a period of complete submergence followed by desubmergence, during seed germination and the vegetative phase of development. The ability of rice to survive intermittent water deficiency requires abscisic acid (ABA) perception and protective response mechanisms primarily described for shoot tissues. Soil moisture is a major determinant of the development and architecture of rice root systems. Many cultivars thrive in waterlogged anaerobic soils due to constitutive development of traits that facilitate aeration. These include emergence of adventitious crown roots from the basal nodes of the shoot, and the formation of gas passages within the cortical layer called aerenchyma, a gas-impermeable apoplastic barrier on exodermal cell walls that contains suberin.

There is increasing incentive in agriculture to develop cultivars with enhanced tolerance to environmental stresses and pathogens. Furthermore, as carbon-rich suberin is produced from carbon fixed during photosynthesis and is inert in soils, there is increasing interest in promoting suberin deposition in root systems as a means of drawdown of atmospheric CO₂ by annual and perennial crops, including long lived tree crops.

Identification of the cis- and trans-regulatory factors that control the progression of development and key metabolic programs in plants can inform breeding and biotechnological efforts to improve climate resilience of rice and other crops. The present disclosure describes such regulatory factors and related advances.

### BRIEF SUMMARY OF THE INVENTION

Alteration of suberized cell wall composition would be a suitable option to improve plant stress tolerance. Since most crop products generally contain less suberin that their stress tolerant wild relative, a method for controlling suberin deposition would be economically valuable.

In some embodiments, the disclosure provides a plant having decreased suberin, wherein the plant ectopically expresses or overexpresses one or more polypeptide that is substantially identical to one or more protein as provided in Table 1 or SEQ ID NOS: 1 or 2, or a polypeptide having at least 70%, 75%, 80%, 85%, 90%, or 95% identity to SEQ ID NO:1 or 2, wherein the plant has decreased suberin compared to a control plant not ectopically expressing or overexpressing the one or more polypeptide. In some embodiments, the disclosure provides a plant having increased suberin, wherein the plant ectopically expresses or overexpresses one or more polypeptides comprising an amino acid sequence having at least 70% identity, or at least 75% idenity, to an amino acid sequence of any one of SEQ ID NOS: 3-20, wherein the plant has increased suberin compared to a control plant not ectopically expressing or overexpressing the one or more polypeptides. In some embodiments, the plant ectopically expresses or overexpresses one or more polypeptides comprising an amino acid sequence having at least 80% identity, or at least 85% identity, to an amino acid sequence of any one of SEQ ID NOS: 3-20. In some embodiments, the plant ectopically expresses or overexpresses one or more polypeptides comprising an amino acid sequence having at least 90% identity, or at least 95% identity, to an amino acid sequence of any one of SEQ ID NOS: 3-20, In some embodiments, the amino acid sequence comprises any one of SEQ ID NOS:3-20.

In some embodiments, the disclosure provides a plant having increased suberin, wherein the plant ectopically expresses or overexpresses one or more polypeptides comprising an amino acid sequence having at least 90% identity, or at least 95% identity, to an amino acid sequence of any one of SEQ ID NOS:3, 6, 7, 8, 9, 10, or 11, wherein the plant has increased suberin compared to a control plant not ectopically expressing or overexpressing the one or more polypeptides. In some embodiments, the amino acid sequence comprises any one of SEQ ID NOS:3, 6, 7, 8, 9, 10, or 11.

In some embodiments, the plant is a Gramineae plant. In some embodiments, the plant is a rice plant. In some embodiments the plant is a monocot other than a Gramineae, a eudicot, or a gymnosperm.

In some embodiments, the plant comprises an expression cassette comprising a promoter operably linked to a polynucleotide encoding one or more polypeptides having at least 90% identity, or at least 95% identity, to the amino acid sequence of any one of SEQ ID NOS: 3-20. In some embodiments, the one or more polypeptides comprise any one of SEQ ID NOS:3-20. In some embodiments, the plant comprises an expression cassette comprising a promoter operably linked to a polynucleotide encoding one or more polypeptides having at least 90% identity, or at least 95% identity, to the amino acid sequence of any one of SEQ ID NOS:3, 6, 7, 8, 9, 10, or 11. In some embodiments, the one or more polypeptides comprises any one of SEQ ID NOS:3, 6, 7, 8, 9, 10, or 11. In some embodiments, the promoter is inducible. In some embodiments, the promoter is a growth stage-specific promoter. In some embodiments, the promoter is a tissue- or cell-specific promoter.

In some embodiments, the disclosure provides a part of the plant as described above or elsewhere herein. In some embodiments, the part of the plant can be a tuber, a fruit, a grain, a root, or other part of the plant as described above or elsewhere herein.

In some embodiments, the disclosure provides a method of making suberin. In some embodiments, the method comprises providing the plant or the part of the plant as described above or elsewhere herein; and extracting suberin from the plant or the part of the plant.

In some embodiments, the disclosure provides a method of cultivating plants that are tolerant to drought or high salinity conditions, the method comprising, cultivating the plant as described above or elsewhere herein under high salinity, drought, or flooding conditions.

In some embodiments, the disclosure provides a plant having increased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptide that is substantially identical to one or more protein as provided in Table 1 or SEQ ID NOS: 1 or 2, wherein the plant has increased suberin compared to a control plant that expresses the one or more polypeptide. In some embodiments, the disclosure provides a plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 90% identity, or at least 95% identity, to an amino acid sequence any one of SEQ ID NOS: 3-20, wherein the plant has decreased suberin compared to a control plant that expresses the one or more polypeptides. In some embodiments, the one or more polypeptides comprise a sequence of any one of SEQ ID NO:3-20.

In some embodiments, the disclosure provides a plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 90% identity, or at least 95% identity, to an amino acid sequence any one of SEQ ID NOS:3, 6, 7, 8, 9, 10, or 11, wherein the plant has decreased suberin compared to a control plant that expresses the one or more polypeptides. In some embodiments, the one or more polypeptides comprise a sequence of any one of SEQ ID NOS:3, 6, 7, 8, 9, 10, or 11.

In some embodiments, the plant is a Gramineae plant. In some embodiments the plant is monocot other than a Gramineae, a eudicot, or a gymnosperm. In some embodiments, the plant is a rice plant.

Other contemplated methods of modulating the level of gene activity include those described below. Other aspects of the invention are disclosed elsewhere herein.

### BRIEF DESCRIPTION OF THE DRAWINGS

FIG. 1. Chromatin accessibility and transcript abundance are coordinately and reversibly regulated in root systems of rice grown under plate, greenhouse and field conditions.
(A) Overview of the ten growth conditions and treatments used in this study of conditional dynamics in gene activity in rice. Water sufficient and extreme treatments were performed on pot-grown plants. (B) Genotype p35S:INTACT was used for nuclei capture to evaluate chromatin accessibility by ATAC-seq. Genotype p35S: TRAP was used for polyadenylated (polyA) mRNA-seq and Translating Ribosome Affinity Purification for TRAP-seq of polyadenylated mRNAs associated with ribosomes. RNA abundance and chromatin accessibility based on transposase hypersensitivity sites (THSs) were integrated in analyses surveying presence of known TF motifs. (C) Volcano plots demonstrate significant changes in chromatin accessibility in response to water deficit (WD) and submergence (SUB) are reversed by 1 d post-stress recovery (+1R). THSs are very similar in 5 d waterlogged (WL) relative to pot-grown control (well-watered) root systems (CON). Differential THSs (dTHSs) are distinguishable between pot and field grown roots. (D) Violin plots demonstrate consistent up or downregulation of polyA RNA (log2 fold change [FC]) for genes with significant up or downregulation in chromatin accessibility over nearby THSs (Padj (adjusted)<0.05) in response to WD and SUB and recovery relative to CON, and in the Field 1285 versus CON comparison. ANOVA was used to evaluate differences in mean log 2 FC; letters indicate groups in an LSD test. (E) Integrated cluster analysis of ATAC-seq and RNA abundance dynamics. Heatmap of log 2 FC of differentially regulated transcripts (|log2 FC|>1 and Padj<0.01]) and log 2 FC of chromatin accessibility within 2 kb upstream of transcript coding regions in response to WD, WL, SUB, or recovery (+1R, plate and field) growth for polyA and ribosome-associated polyA (TRAP) RNA. All comparisons are relative to pot-grown control (CON) root systems. Gene number in cluster in parenthesis. Selected significant Gene Ontology (GO) category enrichment indicated to provide biological context. (F) Genome browser view of chromatin and transcript data for representative genes. Genes are orientated from 5′ to 3′. Scale bar equals 0.5 kb.

FIG. 2. Responses to water deficit and recovery across root domains reveal dynamic 1332 patterns of translatomes and contrasting metabolic pathways. (A and B) Gene activity of cell populations of meristematic regions including dividing cells (pRSS1), quiescent center and metaxylem (pQHB), cortex (pCMZ), and endodermis (pAtSCR) 1335 were contrasted with differentiated regions in endodermis (pCASP3), endodermis/exodermis (pLSI1), and others included in p35S. A heatmap depicts relative TRAP RNA levels for genes with differences in control, water deficit and one-day recovery calculated by dominant pattern analysis, and subsequent enrichment of TF binding sites in THSs located nearby genes in each pattern. Number of genes in pattern in parenthesis. (C) Histochemical staining of rice crown roots for suberin using Fluorol Yellow (FY) in longitudinal views of the exodermis and radial cross sections. Quantification of exodermal FY signal of control and water deprived plants by Corrected Total Cell Fluorescence (CTCF). Asterisks indicate significant differences by Student's t-test (p=0.005). (D) An inferred regulatory network of genes in coexpression module N5:MOD000731 enriched for genes associated with suberin biosynthesis and the pLS1 cell population under water deficit. Large circles indicate TFBSs enriched in proximal THSs; hexagons are TFBSs associated with 1347 TF genes in the coexpression module. (E) A model of dynamics within the pLSI1-marked cells. Briefly, transcripts associated with N assimilation processes and iron uptake are translated in control conditions. In the water deficit translatome, Fe uptake is downregulated and plastidial fatty acid biosynthesis is upregulated, feeding into suberin biosynthesis and the deposition of apoplastic suberin. In the recovery condition, the Fe uptake translatome signature is restored and there is further upregulation of N assimilation genes, including the ferredoxin cofactor which uses Fe in N assimilation.

FIG. 3. (A) Suberin biosynthetic pathway overlaid with genes in N5:MOD000731. Pathway diagram adapted from Vishwanath, S. J., Delude, C., Domergue, F., and Rowland, 0. (2015). Suberin: biosynthesis, regulation, and polymer assembly of a protective extracellular barrier. Plant Cell Rep. 34, 573-586. Shown are genes present in the coexpression module N5:MOD000731. Not all genes in the module were mapped to steps in the pathway. (B) A spatialHeatmap tool was prepared and used to visualize rice translatome data. Transcript levels are rendered in the anatomical region of expression as well as in a heat map next to each promoter name, for ease of comparison. TPM value scales differ between the representative genes of the network presented in 2E.

### DEFINITIONS

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

The phrase “substantial identity” or “substantially identical,” used in the context of two nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, or 75% to the reference sequence. In some embodiments, a sequence is substantially identical to a reference sequence if the sequence has at least 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Embodiments of the present invention provide for nucleic acids encoding polypeptides (and a heterologous promoter operably linked to a polynucleotide encoding the polypeptides) that are substantially identical to any of the proteins in Table 1 or any one of SEQ ID NOS: 1-20.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, I Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10⁻²⁰.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types, or in specific cells of a tissue. A “growth-stage” specific promoter initiates transcription at a particular stage of growth.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “control plant” refers to a plant that can be compared to a plant as described herein to indicate the effect of a mutation or expression of a protein as described herein. An exemplary control plant is a plant that is otherwise identical or substantially identical to a test plant but that lacks the mutation or heterologously-expressed polypeptide or polynucleotide.

### DETAILED DESCRIPTION OF THE INVENTION

The inventors have identified a number of genes and gene products that influence plant production of suberin, described in Table 1.

Accordingly, one or more of the gene products described herein can be overexpressed or ectopically expressed in a plant to result in increased suberin in the plant as a whole or in cells or tissues in which the gene products are expressed. Increased suberin, increased levels of suberin, or enhanced levels of suberin is at least one of an increase in the level of total suberin, an increase in one or more type of suberin molecule, or increased suberin deposition. Alternatively, one or more of the described genes can be mutated or engineered to reduce expression or activity, or eliminate production of, their encoded gene products, thereby reducing or decreasing suberin production in plant cells or tissues in which the genes have been mutated or engineered. Decreased suberin, reduced suberin, or loss of suberin is at least one of a decrease in the level of total suberin, a decrease in one or more type of suberin molecule, or decreased suberin deposition. Alternatively, expression of the gene products can otherwise be reduced, for example antisense or sense suppression of the gene products.

TABLE 1

| | | | | Putative |
|---|---|---|---|---|
| | Corresponding | | Screen used to | relationship with |
| SEQ ID | Gene | Locus Identifier | identify | suberin* |
| 1 | OsBHLH109 | LOC_Os01g67480 | Membership in | Negative TF |
| | | | inversely expressed | regulator |
| | | | pattern | |
| 2 | OsWRKY107 | LOC_Os01g09080 | Membership in | Negative TF |
| | | | inversely expressed | regulator |
| | | | pattern | |
| 3 | ONAC6 | LOC_Os03g42630 | Coexpression | Positive TF regulator |
| 4 | ZOS1-08 | LOC_Os01g32920 | Coexpression | Positive TF regulator |
| 5 | OsWRKY58 | LOC_Os05g45230 | Coexpression | Positive TF regulator |
| 6 | OsWRKY83 | LOC_Os05g50680 | Coexpression | Positive TF regulator |
| 7 | n/a | LOC_Os02g51799 | Coexpression | Positive TF regulator |
| 8 | n/a | LOC_Os06g11780 | Coexpression | Positive TFregulator |
| 9 | n/a | LOC_Os08g37970 | Coexpression | Positive TFregulator |
| 10 | n/a | LOC_Os06g02250 | Coexpression | Positive TF regulator |
| 11 | OsASFT | LOC_Os11g31090 | Coexpression | Positive regulator |
| | | | | (enzyme in pathway) |
| 12 | n/a | LOC_Os05g38350 | Coexpression | Positive regulator |
| | | | | (enzyme in pathway) |
| 13 | n/a | LOC_Os01g63580 | Coexpression | Positive regulator |
| | | | | (enzyme in pathway) |
| 14 | OsLACS5 | LOC_Os01g46750 | Coexpression | Positive regulator |
| | | | | (enzyme in pathway) |
| 15 | n/a | LOC_Os02g44654 | Coexpression | Positive regulator |
| | | | | (enzyme in pathway) |
| 16 | n/a | LOC_Os04g47250 | Coexpression | Positive regulator |
| | | | | (enzyme in pathway) |
| 17 | n/a | LOC_Os08g44360 | Coexpression | Positive regulator |
| | | | | (enzyme in pathway) |
| 18 | n/a | LOC_Os09g39410 | Coexpression | Positive regulator |
| | | | | (enzyme in pathway) |
| 19 | n/a | LOC_Os11g37900 | Coexpression | Positive regulator |
| | | | | (enzyme in pathway) |
| 20 | n/a | LOC_Os04g50680 | TFBS Enrichment | Positive TF regulator |

*Designation as a putative enzyme in the pathway is based on homology, patterns of co-regulation, and conditional regulation in cells that produce suberin. Designation as a putative transcription factor (TF) is based on the presence of DNA binding or other characteristic protein sequence domains. Genes are implicated as regulators based on where and when they are expressed, as well as evidence of regulation of other genes. For example, for evidence as a regulator, Alex identified targets (i.e., enzymes) based on the presence of a DNA binding motif corresponding to the transcriptional regulator in co-expressed genes.
TFBS = TF Binding Sites.

Upregulation (e.g., overexpression or ectopic expression) can result in a variety of beneficial phenotypes, including:
1. Upregulation of transcription factors and enzymes (any and collectively) identified herein will enhance the expression of suberin biosynthetic genes and, as a consequence, increase suberin in the plant. Increased suberin will lead to an improved tolerance of the plant to one or more of the following: drought, flooding or elevated salt concentration in the soil. It will also increase the resistance to plant pathogens. Increased suberin will also lead to greater concentration of carbon in the roots inside the soil, allowing for increased carbon sequestration from the atmosphere into the soil.
2. Ectopic expression of transcription factors and enzymes (any and collectively) identified herein in alternative tissues (for example, but not limited to, tubers, fruits and seeds, stems, roots) will enhance levels of suberin in these tissues, or in specific cell types, for example, but not limited to, exodermis. An increased level of suberin or specific suberin molecules in these will lead to reduced water loss, increased resistance to rotting, and increased shelf life of derived agronomical products, including but not limited to tubers such as potatoes.
3. Upregulation of any one or a combination of transcription factors and enzymes (any and collectively) identified herein will enhance the accumulation of suberin and its specific monomers.

This will provide a low-cost and renewable source for these components, which could later be efficiently extracted, for example but not limited to by chemical methods, and can used in industrial applications. These applications can include, but are not limited to, synthesis of hybrid co-polymers, resins, or fibers. Suberin extracts have also shown medical properties as cancer-preventing anti-mutagenic agents and as a firming anti-wrinkle agent in human skin. Downregulation of suberin can result in a variety of beneficial phenotypes, including:
1. Disruption of suberin (for example but not limited to mutation or engineering of any one or a combination of the genes described herein will lead to partial or total loss of suberin in the plant. The loss of suberin in the root will lead to increased levels of colonization of the plant by beneficial microbes and greater beneficial interaction between plant and soil microbiome.
2. Loss of suberin in the root will alter the morphological and physical properties of the root. These changes can be applied to change the properties of certain roots and tubers, making them more appealing/suitable to human consumption.

Accordingly, the disclosure provides methods of modulating (increase or decrease) suberin levels in a plant by altering expression or activity of a protein substantially identical to one listed in Table 1 or from SEQ ID NOS: 1-20, for example, by introducing into a plant a recombinant expression cassette comprising a regulatory element (e.g., a promoter) operably linked to a polynucleotide encoding the protein.

In some embodiments, the disclosure provides for increasing and/or ectopically expressing one or more of the proteins in a plant. In some embodiments, the disclosure provides a plant having increased suberin, wherein the plant ectopically expresses or overexpresses one or more polypeptides comprising an amino acid sequence having at least 60%, 65%, 70%, 75%, 80%, 85%, 90%, 91%, 92%, 93%, 94%, 95%, 96%, 97%, 98%, or 99% identity to an amino acid sequence of any one of SEQ ID NOS:1-20, or any one of SEQ ID NOS:3-20, wherein the plant has increased suberin compared to a control plant not ectopically expressing or overexpressing the one or more polypeptides. In some embodiments, the plant ectopically expresses or overexpresses one or more polypeptides comprising an amino acid sequence having at least 90% identity to an amino acid sequence of any one of SEQ ID NOS:3-20. In some embodiments, the plant ectopically expresses or overexpresses one or more polypeptides comprising an amino acid sequence having at least 91%, at least 92%, at least 93%, at least 94%, at least 95%, at least 96%, at least 97%, at least 98%, or at least 99% identity to an amino acid sequence of any one of SEQ ID NOS:3-20. In some embodiments, the plant ectopically expresses or overexpresses one or more polypeptides comprising an amino acid sequence comprises any one of SEQ ID NOS:3-20.

In some embodiments, the disclosure provides a plant having increased suberin, wherein the plant ectopically expresses or overexpresses one or more polypeptides comprising an amino acid sequence having at least 60%, 65%, 70%, 75%, 80%, 85%, 90%, 91%, 92%, 93%, 94%, 95%, 96%, 97%, 98%, or 99% identity to an amino acid sequence of any one of SEQ ID NOS:3 and 6-11, wherein the plant has increased suberin compared to a control plant not ectopically expressing or overexpressing the one or more polypeptides. In some embodiments, the plant ectopically expresses or overexpresses one or more polypeptides comprising an amino acid sequence having at least 90% identity, or at least 95% identity, to an amino acid sequence of any one of SEQ ID NOS:3 and 6-11. In some embodiments, the plant ectopically expresses or overexpresses one or more polypeptides comprising an amino acid sequence having at least 91%, at least 92%, at least 93%, at least 94%, at least 95%, at least 96%, at least 97%, at least 98%, or at least 99% identity to an amino acid sequence of any one of SEQ ID NOS:3 and 6-11. In some embodiments, the plant ectopically expresses or overexpresses one or more polypeptides comprising an amino acid sequence comprises any one of SEQ ID NOS:3 and 6-11.

Any of a number of means well known in the art can be used to increase expression or activity in plants. In some embodiments, overexpression and/or ectopical expression of one or more proteins in a plant is through introducing one or more expression cassettes into the plant. In some embodiments, the disclosure provides a plant comprises an expression cassette comprising a promoter operably linked to a polynucleotide encoding one or more polypeptides having at least 60%, 65%, 70%, 75%, 80%, 85%, 90%, 91%, 92%, 93%, 94%, 95%, 96%, 97%, 98%, or 99% identity of the amino acid sequence of any one of SEQ ID NO:1-20, or any one of SEQ ID NOS:3-20. In some embodiments, the plant comprises an expression cassette comprising a promoter operably linked to a polynucleotide encoding one or more polypeptides having at least 90% identity, or at least 95% identity, to the amino acid sequence of any one of SEQ ID NO:3-20. In some embodiments, the plant comprises an expression cassette comprising a promoter operably linked to a polynucleotide encoding one or more polypeptides having at least 91%, at least 92%, at least 93%, at least 94%, at least 95%, at least 96%, at least 97%, at least 98%, or at least 99% identity to an amino acid sequence of any one of SEQ ID NO:3-20. In some embodiments, the plant comprises an expression cassette comprising a promoter operably linked to a polynucleotide encoding an amino acid sequence of any one of SEQ ID NO:3-20.

In some embodiments, the disclosure provides a plant comprises an expression cassette comprising a promoter operably linked to a polynucleotide encoding one or more polypeptides having at least 60%, 65%, 70%, 75%, 80%, 85%, 90%, 91%, 92%, 93%, 94%, 95%, 96%, 97%, 98%, or 99% identity of the amino acid sequence of any one of SEQ ID NO:3 and 6-11. In some embodiments, the plant comprises an expression cassette comprising a promoter operably linked to a polynucleotide encoding one or more polypeptides having at least 90% identity of the amino acid sequence of any one of SEQ ID NO:3 and 6-11. In some embodiments, the plant comprises an expression cassette comprising a promoter operably linked to a polynucleotide encoding one or more polypeptides having at least 91%, at least 92%, at least 93%, at least 94%, at least 95%, at least 96%, at least 97%, at least 98%, or at least 99% identity to an amino acid sequence of any one of SEQ ID NO:3 and 6-11. In some embodiments, the plant comprises an expression cassette comprising a promoter operably linked to a polynucleotide encoding an amino acid sequence of any one of SEQ ID NO:3 and 6-11.

In some embodiments, the promoter is inducible. In some embodiments, the promoter is a growth stage-specific promoter. In some embodiments, the promoter is a tissue- or cell-specific promoter. Alternatively, one or several genes can be expressed constitutively (e.g., using the CaMV 35S promoter or other constitutive promoter). In some embodiments, selective promoters are used to drive expression as discussed further below.

Where enhanced expression of a gene is desired, the desired gene (or at least the polynucleotide encoding the protein) from the same species or a different species (or substantially identical to the gene or polynucleotide encoding the protein from another species) may be used. In some embodiments, to decrease potential sense suppression effects, a polynucleotide from a different species (or substantially identical to the gene or polynucleotide from another species) may be used.

Any of a number of means well known in the art can be used to increase expression or activity in plants. In some embodiments, the plant is a Gramineae plant. In some embodiments, the plant is a rice plant. In some embodiments the plant is monocot other than a Gramineae, a eudicot, or a gymnosperm. Any organ or plant part can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat), fruit, abscission zone, etc. In some embodiments, the disclosure provides a part of the plant. In some embodiments, the part of the plant can be a tuber, a fruit, a grain, a root, or other part of the plant as described above or elsewhere herein.

One of skill will recognize that the polypeptides, like other proteins, can have different domains which perform different functions. Thus, the overexpressed or ectopically expressed polynucleotide sequences need not be full length, so long as the desired functional domain of the protein is expressed. Alternatively, or in addition, active proteins can be expressed as fusions, without necessarily significantly altering activity. Examples of fusion partners include, but are not limited to, poly-His or other tag sequences.

Alternatively, expression or activity of the proteins described herein can be reduced or inhibited. Any one or more of the genes provided in Table 1 can be knocked out, mutated, or engineered to decrease suberin in a plant or plant cell. For example, in some embodiments, the native gene sequence mutated, knocked out, or engineered in a plant encodes a polypeptide identical or substantially identical (e.g., at least 70, 75, 80, 85, 90, or 95% identical) to a protein of Table 1 or of any one of SEQ ID NO: 1-20. Gene sequences can be readily identified in many plant species in view of known genome sequences and the conserved nature of the proteins.

In some embodiments, the disclosure provides a plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 60%, 65%, 70%, 75%, 80%, 85%, 90%, 91%, 92%, 93%, 94%, 95%, 96%, 97%, 98%, or 99% identity to an amino acid sequence any one of SEQ ID NOS:3-20, or any one of SEQ ID NOS: 1-20, wherein the plant has decreased suberin compared to a control plant that expresses the one or more polypeptides. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 90% identity, or at least 95% identity, to an amino acid sequence any one of SEQ ID NOS:3-20. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 91%, at least 92%, at least 93%, at least 94%, at least 95%, at least 96%, at least 97%, at least 98%, or at least 99% identity to an amino acid sequence of any one of SEQ ID NOS:3-20. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising an amino acid sequence comprises any one of SEQ ID NOS:3-20.

In some embodiments, the disclosure provides a plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 60%, 65%, 70%, 75%, 80%, 85%, 90%, 91%, 92%, 93%, 94%, 95%, 96%, 97%, 98%, or 99% identity to an amino acid sequence any one of SEQ ID NOS:3 and 6-11, wherein the plant has decreased suberin compared to a control plant that expresses the one or more polypeptides. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 90% identity to an amino acid sequence any one of SEQ ID NOS:3 and 6-11. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 91%, at least 92%, at least 93%, at least 94%, at least 95%, at least 96%, at least 97%, at least 98%, or at least 99% identity to an amino acid sequence of any one of SEQ ID NOS:3 and 6-11. In some embodiments, the plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising an amino acid sequence comprises any one of SEQ ID NOS:3 and 6-11.

In some embodiments, the gene sequence is knocked out in the plant. “Knocked out” means that the plant does not make the particular protein encoded by the gene. Knockouts can be achieved in a variety of ways. For the purposes of this document, a knock out can be achieved by a deletion of all or a substantial part (e.g., majority) or the coding sequence for a polypeptide identical or substantially identical to a protein of Table 1 or any one of SEQ ID NO: 1-20. Alternatively a knock out can be achieved by introduction of a mutation that prevents translation or transcription (e.g., a mutation that introduces a stop codon early in the coding sequence or that disrupts transcription). A knock out can also be achieved by silencing or other suppression methods, e.g., such that the plant expresses substantially less of the native protein (e.g., less than 50, 25, 10, 5, or 1% of native expression).

In some embodiments, the mutation introduced into the protein is a single amino acid change that reduces or eliminates the protein's activity. Alternatively, the mutation can include any number (e.g., 1, 2, 3, 4, 5, 6, 7, 8, 9, or 10 or more) of amino acid changes, deletions or insertions that reduce or eliminate the protein activity.

Methods for introducing genetic mutations into plant genes and selecting plants with desired traits are well known and can be used to introduce mutations or to knock out a protein. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having mutated protein can then be identified, for example, by phenotype or by molecular techniques.

Modified protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski et al., Meth. Enzymol., 194:302-318 (1991)). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications or knockouts by specifically targeting the gene in vivo (see, generally, Grewal and Klar, Genetics, 146:1221-1238 (1997) and Xu et al., Genes Dev., 10:2411-2422 (1996)). Homologous recombination has been demonstrated in plants (Puchta et al., Experientia, 50:277-284 (1994); Swoboda et al., EMBO 1, 13:484-489 (1994); Offringa et al., Proc. Natl. Acad. Sci. USA, 90:7346-7350 (1993); and Kempin et al., Nature, 389:802-803 (1997)).

In applying homologous recombination technology to a gene, mutations in selected portions of gene sequences (including 5′ upstream, 3′ downstream, and intragenic regions) can be made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA, 91:4303-4307 (1994); and Vaulont et al., Transgenic Res., 4:247-255 (1995) are conveniently used to increase the efficiency of selecting for altered PP2A subunit A protein gene expression in transgenic plants. The mutated gene will interact with the target wild-type gene in such a way that homologous recombination and targeted replacement of the wild-type gene will occur in transgenic plant cells, resulting in suppression of target protein activity.

Any of a number of genome editing proteins known to those of skill in the art can be used to mutate or knock out the target protein. The particular genome editing protein used is not critical, so long as it provides site-specific mutation of a desired nucleic acid sequence. Exemplary genome editing proteins include targeted nucleases such as engineered zinc finger nucleases (ZFNs), transcription-activator like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 endonuclease to the target cleavage site.

CRISPR (Clustered Regularly Interspaced Short Palindromic Repeats)/Cas (CRISPR-associated) system, are adaptive defense systems in prokaryotic organisms that cleave foreign DNA. CRISPR loci in microbial hosts contain a combination of CRISPR-associated (Cas) genes as well as non-coding RNA elements which determine the specificity of the CRISPR-mediated nucleic acid cleavage. Three types (I-III) of CRISPR systems have been identified across a wide range of bacterial hosts. In the typical system, a Cas endonuclease (e.g., Cas9) is guided to a desired site in the genome using small RNAs that target sequence-specific single- or double-stranded DNA sequences. The CRISPR/Cas system has been used to induce site-specific mutations in plants (see Miao et al. 2013 Cell Research 23:1233-1236).

The basic CRISPR system uses two non-coding guide RNAs (crRNA and tracrRNA) which form a crRNA:tracrRNA complex that directs the nuclease to the target DNA via Wastson-Crick base-pairing between the crRNA and the target DNA. Thus, the guide RNAs can be modified to recognize any desired target DNA sequence. More recently, it has been shown that a Cas nuclease can be targeted to the target gene location with a chimeric single-guide RNA (sgRNA) that contains both the crRNA and tracRNA elements. It has been shown that Cas9 can be targeted to desired gene locations in a variety of organisms with a chimeric sgRNA (Cong et al. 2013 Science 339:819-23).

As described in the addgene.org website, defective (non-cleaving) Cas9 or other nucleases that are fused to VP16 or other transcriptional activation domain can also be used with a gRNA to promote the upregulation of a gene. There are constructs that can do the same to promote the downregulation of specific genes.

Zinc finger nucleases (ZFNs) are engineered proteins comprising a zinc finger DNA—binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see Urnov et al. 2010 Nat Rev Genet. 11(9):636-46.

Transcription activator like effectors (TALEs) are proteins secreted by certain species of Xanthomonas to modulate gene expression in host plants and to facilitate bacterial colonization and survival. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site have been found. The simple relationship between amino acid sequence and DNA recognition of the TALE binding domain allows for the design DNA binding domains of any desired specificity.

TALEs can be linked to a non-specific DNA cleavage domain to prepare genome editing proteins, referred to as TALENs. As in the case of ZFNs, a restriction endonuclease, such as FokI, can be conveniently used. For a description of the use of TALENs in plants, see Mahfouz et al. 2011 Proc Natl Acad Sci USA. 108:2623-8 and Mahfouz 2011 GM Crops. 2:99-103.

Meganucleases are endonucleases that have a recognition site of 12 to 40 base pairs. As a result, the recognition site occurs rarely in any given genome. By modifying the recognition sequence through protein engineering, the targeted sequence can be changed and the nuclease can be used to cleave a desired target sequence. (See Seligman, et al. 2002 Nucleic Acids Research 30: 3870-9 WO06097853, WO06097784, WO04067736, or US20070117128).

In addition to the methods described above, other methods for introducing genetic mutations into plant genes and selecting plants with desired traits are known. For instance, seeds or other plant material can be treated with a mutagenic chemical substance, according to standard techniques. Such chemical substances include, diethyl sulfate, ethylene imine, ethyl methanesulfonate (EMS) and N-nitroso-N-ethylurea. Alternatively, ionizing radiation from sources such as, X-rays or gamma rays can be used.

Also provided are methods of suppressing expression or activity of a polypeptide substantially identical to a protein of Table 1 or any one of SEQ ID NOS: 1-20 in a plant using expression cassettes that RNA molecules (or fragments thereof) that inhibit endogenous target expression or activity in a plant cell. Suppressing or silencing gene function refers generally to the suppression of levels of mRNA or protein expressed by the endogenous gene and/or the level of the protein functionality in a cell. The terms do not require specific mechanism and could include RNAi (e.g., short interfering RNA (siRNA) and microRNA (miRNA)), anti-sense, cosuppression, viral-suppression, hairpin suppression, stem-loop suppression, and the like.

A number of methods can be used to suppress or silence gene expression in a plant. The ability to suppress gene function in a variety of organisms, including plants, using double stranded RNA is well known. Expression cassettes encoding RNAi typically comprise a polynucleotide sequence at least substantially identical to the target gene linked to a complementary polynucleotide sequence. The sequence and its complement are often connected through a linker sequence that allows the transcribed RNA molecule to fold over such that the two sequences hybridize to each other.

RNAi (e.g., siRNA, miRNA) appears to function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides in length that are processed from longer precursor transcripts that form stable hairpin structures.

In addition, antisense technology can be conveniently used. To accomplish this, a nucleic acid segment at least substantially identical to the desired gene is cloned and operably linked to a promoter such that the antisense strand of RNA will be transcribed. The expression cassette is then transformed into a plant and the antisense strand of RNA is produced. In plant cells, it has been suggested that antisense RNA inhibits gene expression by preventing the accumulation of mRNA which encodes the protein of interest.

Another method of suppression is sense suppression. Introduction of expression cassettes in which a nucleic acid is configured in the sense orientation with respect to the promoter has been shown to be an effective means by which to block the transcription of target genes.

For these techniques, the introduced sequence in the expression cassette need not have absolute identity to the target gene. In addition, the sequence need not be full length, relative to either the primary transcription product or fully processed mRNA. One of skill in the art will also recognize that using these technologies families of genes can be suppressed with a transcript. For instance, if a transcript is designed to have a sequence that is conserved among a family of genes, then multiple members of a gene family can be suppressed. Conversely, if the goal is to only suppress one member of a homologous gene family, then the transcript should be targeted to sequences with the most variance between family members.

Gene expression can also be inactivated using recombinant DNA techniques by transforming plant cells with constructs comprising transposons or T-DNA sequences. Mutants prepared by these methods are identified according to standard techniques. For instance, mutants can be detected by PCR or by detecting the presence or absence of PP2A subunit A mRNA, e.g., by northern blots or reverse transcription PCR (RT-PCR).

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs. The design and use of target RNA-specific ribozymes is well known.

The recombinant construct encoding a genome editing protein or a nucleic acid that suppresses expression may be introduced into the plant cell using standard genetic engineering techniques, well known to those of skill in the art. In the typical embodiment, recombinant expression cassettes can be prepared according to well-known techniques. In the case of CRISPR/Cas nuclease, the expression cassette may transcribe the guide RNA, as well.

In some embodiments, the genome editing protein itself, is introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell.

In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

Plant expression cassettes (e.g., for expression of the proteins described herein, or alternatively for expression of siRNA or gene editing proteins) can contain the polynucleotide operably linked to a promoter (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used. A plant promoter fragment can be employed which will direct expression of the desired polynucleotide in all tissues of a plant. In some embodiments, promoters described herein comprise 2 kb region upstream (5′) from where gene transcription is initiated.

Such promoters are referred to herein as “constitutive” promoters and are active under most environmental conditions and state of development or cell differentiation. Examples of constitutive promoters include the cauliflower mosaic virus (CaMV) 35S transcription initiation region.

Alternatively, the plant promoter can direct expression of the polynucleotide under environmental control. Such promoters are referred to here as “inducible” promoters. Environmental conditions that may affect transcription by inducible promoters include biotic stress, abiotic stress, saline stress, drought stress, pathogen attack, flooding/anaerobic conditions, cold stress, heat stress, hypoxia stress, or the presence of light.

In addition, chemically inducible promoters can be used. Examples include those that are induced by benzyl sulfonamide, tetracycline, abscisic acid, dexamethasone, ethanol or cyclohexenol.

Examples of promoters under developmental control include promoters that initiate transcription only, or preferentially, in certain tissues such as leaves, roots, fruit, seeds, or flowers. These promoters are sometimes called tissue-preferred promoters. The operation of a promoter may also vary depending on its location in the genome. Thus, a developmentally regulated promoter may become fully or partially constitutive in certain locations. A developmentally regulated promoter can also be modified, if necessary, for weak expression.

In some embodiments, the promoter directs expression in the exodermis, endodermis, phellem, or a sub-combination or combination of these. These are internal root tissues of the plant. Enhancing suberin in one or more of these tissues, can in some embodiments enhance tolerance to stresses and pathogens. Additionally, expression of suberin-enhancing proteins under the control of a phellem promoter can be used to improve tuber quality.

In some embodiments, the promoter directs expression in fruit epidermis. Such promoters can be used for expressing suberin-promoting genes in the epidermis of fruits to fortify the cuticule and reduce water loss, increase resistance to rotting, and increase shelf life of fruits.

Additional exemplary promoters include but are not limited to the following:

Exemplary Drought-Responsive Exodermis-Enriched Promoters:
OS01G010OS00, OS01G0150000, OS01G0252300, OS01G0252400, OS01G0269800, OS01G0315600, OS01G0315700, OS01GOS12700, OS01G0595201, OS01G0601200, OS01G0607000, OS01G0615500, OS01G0621500, OS01G0636400, OS01G0677400, OS01G0718500, OS01G0761400, OS01G0827300, OS01G0836600, OS01G0850550, OS01G0854800, OS01G0855000, OS01G0871100, OS01G0871150, OS01G0891000, OS01G0891033, OS01G0917700, OS01G0917801, OS01G0924933, OS01G0939600, OS02G0114300, OS02G0114400, OS02G0136900, OS02G0179500, OS02G0189200, OS02G0276400, OS02G0302900, OS02GOS03500, OS02G0547300, OS02G0607700, OS02G0653200, OS02G0666500, OS02G0666550, OS02G0706900, OS02G0710201, OS02G0710300, OS02G0715000, OS02G0727300, OS02G0727400, OS02G0732800, OS02G0752600, OS02G0754400, OS02G0754450, OS02G0820200, OS02G0823900, OS03G0220100, OS03G0220200, OS03G0233200, OS03G0233251, OS03G0245500, OS03G0245700, OS03G0257500, OS03G0261100, OS03G0281900, OS03G0297900, OS03G0304100, OS03G0339400, OS03G0359600, OS03G0576500, OS03G0624600, OS03G0624700, OS03G0664400, OS03G0664600, OS03G0664650, OS03G0694500, OS03G0694600, OS03G0782400, OS03G0793800, OS03G0793900, OS03G0793950, OS03G0794000, OS03G0849800, OS03G0850700, OS01G0106500, OS01G0107300, OS04G0115500, OS01G0165000, OS01G0165100, OS01G0271200, OS01G0382900, OS04G0423800, OS01G0439600, OS01G0456300, OS01G0483500, OS01G0483550, OS04GOS06300, OS01G0521900, OS01G0533900, OS01G0544200, OS01G0545400, OS04G0545600, OS01G0560100, OS01G0590100, OS01G0609300, OS01G0609400, OS04G0627300, OS01G0661100, OS05G0226900, OS05G0245300, OS05G028OS00, OS05G0280600, OS05G0304701, OS05G0355700, OS05G0355900, OS05G0384600, OS05G0457800, OS05G0528500, OS05G0577100, OS05G0577150, OS05G0583600, OS06G0112700, OS06G0128100, OS06G0128150, OS06G0132500, OS06G0132550, OS06G0179000, OS06G0221000, OS06G0229400, OS06G0257600, OS06G0274800, OS06G0274875, OS06G0539500, OS06G0594600, OS06G0594650, OS06G0594700, OS07G0101000, OS07G0142200, OS07G0142300, OS07G0142500, OS07G0142600, OS07G0175500, OS07G0175600, OS07G0475000, OS07G0497500, OS07G0524200, OS07G0599300, OS07G0599500, OS07G0599600, OS07G0599700, OS07G0599900, OS07G0600200, OS07G0612400, OS07G0618400, OS07G0625800, OS07G0668300, OS07G0677600, OS08G0162650, OS08G0162700, OS08G0280600, OS08G0335600, OS08G0478000, OS08G0486300, OS08G0486400, OS08G0492000, OS09G0127300, OS09G0488800, OS09G0567500, OS09G0567600, OS10G0155700, OS01G0413400, OS10G0438600, OS10G0438800, OS10G0479700, OS10G0486000, OS10G0486100, OS10G0532200, OS11G0152025, OS11G0156300, OS11G0466300, OS11G0499600, OS11GOS01000, OS11GOS07200, OS11G0521000, OS11G0546600, OS11G0591200, OS11G0591300, OS11G0600900, OS11G0614800, OS12G0133500, OS12G015OS00, OS12G0151750, OS12G0451300, OS12G0453500, OS12GOS11200, OS12G0566600, OS12G0578300, OS12G0599800, OS12G0610800, OS12G0619000, OS12G0630600.

### Exemplary Core Exodermis-Enriched Promoters

OS05G0405500, OS01G0781100, OS08G0455600, OS03G0802400, OS10G0422300, OS01G0622300, OS02G0101100, OS02G0722450, OS11G0123400, OS05G0162350, OS06G0714200, OS08G0190100, OS11G0129800, OS07G0680000, OS03G0188200, OS06G0107800, OS02G0456500, OS06G0136900, OS01G0452700, OS07G0257400, OS10G0390800, OS07G0533850, OS06G0499500, OS11G0490100, OS07G0142700, OS07G0184900, OS01G0591300, OS01G0818700, OS11G0226700, OS02G0130300, OS01G0786601, OS08G0440800, OS08G0109100, OS01G0551800, OS10G0522000, OS06G0230201, OS01G0639800, OS05G0447500, OS02G0779400, OS01G0775200, OS10G0561250, OS01G0618500, OS11G0490300, OS01G0618950, OS04G0626000, OS01GOS08100, OS01G0128700, OS02G0753700, OS03G0405500, OS12G0293100, OS01G0574550, OS09G0560450, OS01G0525800, OS02G0608100, OS05G0405600, OS02G0280400, OS03G0646350, OS07G0421300, OS06G0719600, OS05G0551000, OS05G0564000, OS07G0530100, OS06G0162500, OS02G0291400, OS03G0199OS0, OS01G0778800, OS02G0231600, OS01G0841600, OS11G0471200, OS04G0664900, OS06G0283300, OS07G0273600, OS01G0917900, OS01G0639200, OS03G0374901, OS07G0181750, OS06G0158OS0, OS11G0169100, OS08G0109150, OS01G0266500, OS02GOS04800, OS11G0223201, OS02G0786450, OS11G0222900, OS05G0499800, OS01G0956300, OS09G0123500, OS01G0193600, OS11G0148700, OS01G0825700, OS08G0197000, OS09G0114700, OS05G0263100, OS01G0106500, OS10G039OS00, OS09G0311600, OS01G0169666, OS12G0170600, OS01G0185200, OS05G0366600, OS02G0755200, OS02G0254233, OS01G0586600, OS11G0160700, OS08G0158400, OS03G0829466, OS09G0570150, OS07G0165200, OS01G0235900, OS09G0551251, OS09G0434900, OS01G0383100, OS01G0788400, OS02G0242100, OS04G0817650, OS01G0469500, OS01G0330200, OS02GOS16600, OS02G0582900, OS02G0820800, OS02G0108800, OS02G0820200, OS06G0622300, OS02G0823100, OS04G0623500, OS08G0455700, OS05G0302300, OS01G0948200, OS02G0163500, OS11G0124300, OS11G0162900, OS01G0634300, OS08G0337700, OS10G0413400, OS09G0533100, OS02G0202900, OS06G0704400, OS03G0642900, OS05G0163700, OS05G0469600, OS03G0773100, OS01G0629300, OS09G0555700, OS01G0477200, OS01G0952700, OS01G0537600, OS09G0525400, OS07G0113700, OS08G0161500, OS02G0822900, OS06G0355150, OS08G0289400, OS01G0664950, OS07G0151800, OS06G0222800, OS12G0442800, OS11G0661400, OS10G0532100, OS07G0434500, OS01G0886900, OS05G0582800, OS12G0635700, OS07G0668000, OS01G0136400, OS08G0542700, OS08G0124500, OS09 G0293400, OS08G0375900, OS02G0656300, OS09G0307800, OS05G0398000, OS12G0582900, OS11G0622800, OS01G0549200, OS09G0332600, OS01G0465800, OS07G0534700, OS06G0301100, OS06G0211500, OS07G0108350, OS02G0601350, OS09 G0249000, OS09G0483700, OS07G0257200, OS07G0280200, OS12G0482500, OS01G0291100, OS12G0101901, OS06G0251200, OS03G0190800, OS08G0159100, OS08G0302000, OS10G0112100, OS01G0120400, OS04G0656100, OS08G0411800, OS11G0575500, OS05G0359500, OS07G0162900, OS07G0459400, OS01G0468600, OS01G0549500, OS06G0158900, OS03G0608800, OS08G0556000, OS02G0788600, OS01G0678800, OS02G0572600, OS01G0647800, OS11G0245100, OS01G0528800, OS02G0574500, OS02G0752000, OS07G0101200, OS01G0822200, OS07G0614300, OS05G0566200, OS01G0327600, OS02G0254100, OS06G0297800, OS05G0359600, OS02G0753800, OS08G0458200, OS09G0557900, OS05G0154600, OS03G0601100, OS07G0479100, OS01G0608300, OS06G0714700, OS02G0214766, OS07G0553700, OS01G0536400, OS12G0634000, OS02G0802500, OS01G0824800, OS01G0629600, OS12G0160900, OS07G0532800, OS07G0556300, OS03G0651300, OS01G0901500, OS07G0673801, OS03G0120900, OS01G0580100, OS07G0119000, OS10G0522200, OS06G0146800, OS03G0289100, OS10G0341750, OS01G0201200, OS01G0525850, OS01GOS17350, OS01G0235850, OS08G0428900, OS07G0658100, OS11G0245200, OS09G0281800, OS02G0682500, OS06G0227200, OS05G0132100, OS11G0686500, OS11G0199700, OS09G0332700, OS02G065OS00, OS04G0690400, OS05G0369500, OS01G0315850, OS06G0189600, OS08G0108700, OS03G0570800, OS02G0281000, OS01G0347600, OS06G0667100, OS01G0708500, OS12G0294100, OS01G0525900, OS07G0123900, OS10G0560450, OS01G074OS00, OS05G0426350, OS12G0197100, OS07G0659400, OS07G0122350, OS07G0176500, OS05G0364650, OS01G0221100, OS02G0193732, OS03G0372600, OS03G0317300, OS08G0440850, OS01G0949800, OS01G0615500, OS06G0711100, OS05G0196500, OS01G0818000, OS06G0259000, OS02G0302900, OS01G0866800, OS06G0719500, OS07GOS16400, OS10G0438600, OS06G0712400, OS11G0123500, OS02G0681900, OS02G0186400, OS01G0219300, OS11G0200950, OS02G0147600, OS01G0599100, OS06G0271600, OS05G0274300, OS06G0247651, OS02G0273100, OS02G0557000, OS03G0198400, OS03G0667300, OS07GOS08101, OS01G0595300, OS01G0648775, OS02G0176700, OS02G0273700, OS03G0582200, OS08G0189850, OS12G0145400, OS08G0424700, OS07G0691600, OS08G0100150, OS07G0673700, OS09 G0455000, OS08G0459100, OS01G0610800, OS01G0162500, OS02G0194900, OS09 G0249100, OS07G0679000, OS03G0266700, OS08G0345700, OS12G0637800, OS03G0391100, OS11G0444700, OS02G0823900, OS05G0117600, OS06G0102100, OS01G0788451, OS03G0401200, OS02G0745100, OS11G0225300, OS06G0724000, OS12G0101800, OS06G0104900, OS01G0148500, OS02G0125200, OS08G0542000, OS01G0877300, OS12G0121300, OS12G0539750, OS08G0542800, OS12G0245100, OS09G0297000, OS09 G0489800, OS03G0570775, OS08G0162700, OS01GOS18300, OS07G0613600, OS05G0241300, OS11G0148400, OS12G0620733, OS03G0194100, OS06G0620700, OS01G0748200, OS12G0565200, OS10GOS06100, OS12G0141600, OS08G0138500, OS01G0812200, OS07G0586900, OS01G0136502, OS07G0175300, OS01G0389100, OS01G0855000, OS03G0669800, OS08G0480200, OS08G0400300, OS08G0327100, OS11G0572301, OS01G0949750, OS12G0620600, OS10GOS16350, OS05G0596500, OS03G0360800, OS02G0130900, OS09G0570000, OS01G0187400, OS06G0115600, OS06G0706000, OS09G0392000, OS01G0877900, OS08G018OS00, OS09G0293500, OS01G0569300, OS03G0427100, OS08G0372900, OS01G0952200, OS01G0574600, OS02G0761150, OS11G0102200, OS01G0130100, OS07G0162700, OS02G0722400, OS06G0632200, OS05G0426400, OS12G0121400, OS01G0751300, OS01G0347500, OS01G0136850, OS10G0561200, OS02G0552100, OS02G0564700, OS01G0111900, OS12G0472500, OS06G0158000, OS11G0252400, OS11G0223300, OS01G0644900, OS10G0316400, OS07G0185700, OS12G0122000, OS03G0771100, OS11G0346016, OS07G0181700, OS03G0359700, OS02GOS11901, OS01GOS12900, OS07GOS16700, OS02G0194800, OS06G0691800, OS07G0126100, OS08G0554400, OS03G0843300, OS01G0214800, OS01G0928800, OS01G0803900, OS12G0172500, OS03G0374900, OS07G0542400, OS08G0162650, OS07G0211101, OS01G0799900, OS08G0398300, OS10G0362700, OS06G0633100, OS12G0636300, OS02G0224100, OS06G0700550, OS04G0634400, OS02G0818800, OS02G0585700, OS01G0859300, OS01G0467100, OS09G0561000, OS03G0829200, OS01G0692800, OS10G0163100, OS01G0209450, OS01G0716800, OS03G0360750, OS02G0535901, OS08G0157800, OS08G0530300, OS08G0554501, OS01G0784900, OS05G0496500, OS12G0484700, OS01G0559550, OS02G0550900, OS01G0523812, OS12G0535300, OS06G0724900, OS01G0598200, OS07G0190900, OS01G0850400, OS05G0196100, OS07G0570100, OS03G0111500, OS01G0495900, OS01G0901700, OS01G0615900, OS01G0689451, OS05G0158600, OS09G0539500, OS09 G0431300, OS01G0189800, OS02G0539500, OS11G0488500, OS04G0266900, OS11G0186800, OS05G0358101, OS11G0249900, OS05G0145700, OS12G0277000, OS01G0822800, OS01G0956200, OS08G0486300, OS01G0548600, OS07G0241700, OS06G0608550, OS12G0183200, OS09G0570100, OS06G0560000, OS09G0489500, OS06G0714600, OS07G0533800, OS12G0127200, OS06G0114651, OS12G0121150, OS11G0117700, OS07G0147700, OS07G0176400, OS05G0359000, OS08G0486400, OS02G0202800, OS01G078OS00, OS03G0616300, OS06G0206900, OS03G0434400, OS02G0823800, OS03G0189400, OS08G0124533, OS07G0101000, OS05G0479700, OS06G0206100, OS09G0454900, OS03G0674900, OS12G0636200, OS02G0639700, OS11G0668100, OS03G0364400, OS01G0873800, OS09G0315800, OS04G0786700, OS02G0651000, OS03G0231000, OS08G0562300, OS01G0880200, OS01GOS10200, OS03G0595600, OS03G0344650, OS05G0490700, OS02GOS04700, OS01G0254100, OS12G0610800, OS03G0839900, OS11G0275000, OS10G0475900, OS07G0530400, OS08G0203900, OS01G0817633, OS08G0482700, OS06G0706100, OS04G0169500, OS07G0162600, OS02G0586800, OS06G0499550, OS06G0630300, OS05G0143300, OS08G0254500, OS07G0280600, OS01G0698300, OS01G0485400, OS07G0125201, OS06G0236300, OS03G0440200, OS02G0152200, OS03G0625800, OS06G0151300, OS01G0232450, OS11G0155600, OS08G0189700, OS06G0199000, OS01G0771900, OS02G0303000, OS03G0646300, OS05G0168200, OS11G0264500, OS05G0162500, OS03G0395300, OS03G0728900, OS11G0146500, OS06G0691700, OS05G0542732, OS09 G0563700, OS08G0356500, OS07G0674700, OS01G0567500, OS04G0151900, OS02G0669366, OS07G0230700, OS09G028OS00, OS05G0578500, OS07G0605200, OS08G0198000, OS06G0603600, OS03G0681400, OS02G0177900, OS01G0195250, OS01G0634900, OS01G0655800, OS09 G0558000, OS01G0779300, OS10G0539200, OS01G0231800, OS01G0648800, OS01G0921800, OS01G0736500, OS09G0536200, OS01G0290800, OS06G0154200, OS09 G0561500, OS02G0585650, OS11G0578100, OS12G0574700, OS01G0394300, OS02G0790600, OS02G0669500, OS07G0230600, OS05G0384600, OS07G0448400, OS02G0527900, OS03G0107300, OS09G0327100, OS02G0788700, OS02G0281101, OS01G0952800, OS01G0639700, OS04G0589700, OS09G0327150, OS02G0629200, OS03G0185700, OS07G0252000, OS07G0177100, OS09G0272200, OS05G0165500, OS06G0102200, OS10G0565100, OS05G0488800, OS01G0949700, OS12G0578300, OS05G0524200, OS01G0688300, OS01G0952300, OS08G0338200, OS10GOS16300, OS03G0241600, OS06G0114650, OS11G0432900, OS06G0221000, OS12G0639200, OS03G0650700, OS03G0776000, OS03G0248600, OS10G0554200, OS01G0870100, OS08G0424600, OS09GOS17700, OS07G0108300, OS02GOS11800, OS10G0363300, OS01G0581700, OS03G0367000, OS04G0623550, OS03G084OS00, OS08G0100200, OS01G0794900, OS06G0608500, OS03G0238300, OS06G0354500, OS08G0254600, OS01G0960400, OS02G0120100, OS06G0151100, OS09G037OS00, OS02G0178000, OS01G0803200, OS01G0128001, OS08G0424100, OS09 GOS11600, OS05G0115800, OS02G0761100, OS06G0559900, OS08G0260600, OS11G0113200, OS07G0449100, OS07G0126500, OS01G0130000, OS11G0595200, OS01G0622800, OS01G0521001, OS07GOS02900, OS06G0151025, OS08G0103000, OS01G0880250, OS06G0134800, OS05G0198400, OS03G0326200, OS06G0187950, OS01G0185900, OS07G0452400, OS07G0532866, OS06G0184400, OS06G0105400, OS06G0142625, OS07G0249700, OS02G0740600, OS03G0802500, OS03G0160400, OS11G021OS00, OS02G0832600, OS06G0720900, OS05G0176300, OS03G0107000, OS01G0948600, OS06G0273700, OS07G0241751, OS08G0563200, OS02GOS04850, OS02G067OS00, OS11G0160300, OS06G0524500, OS09 G0363800, OS01G0765400, OS05G0180600, OS07G0690900, OS02G0629266, OS06G0700601, OS07G0233200, OS06G0221300, OS12G0149900, OS06G0692600, OS07G0625400, OS10G0105700, OS07G0496300, OS06G0111400, OS08G0417000, OS05G0157600, OS01G0965300, OS01G0438400, OS05G0529400, OS05G0560300, OS01G0529100, OS03G0225700, OS05G0469701, OS01G0136800, OS05G0196800, OS02G0754450, OS06G0128300, OS12G0640600, OS01G0630800, OS02G0663900, OS02G0786400, OS03G0145800, OS01G0815400, OS08G0376600, OS06G0247500, OS06G0707300, OS03G0597600, OS02G0578366, OS06G0347700, OS01G0316100, OS01G0794800, OS07G0679OS0, OS01G0521100, OS08G0398800, OS06G0531600, OS03G0255000, OS02G0802575, OS01G0827600, OS05G0358100, OS01G0747750, OS06G0585950, OS04G0617700, OS03G0156700, OS06G0673500, OS02G0312500, OS03G0777700, OS03G0204900, OS07G0683600, OS01G0111400, OS02G0742100, OS02G0807000, OS04G0615800, OS01G0209300, OS07G0466200, OS11G0130400, OS06G0256500, OS03G0851900, OS10G0544600, OS08G0398000, OS07G0668900, OS01G0248500, OS07G0448100, OS01G0283300, OS01G0560100, OS06G0367500, OS01G0811300, OS03G0341000, OS01G0765600, OS05G0440201, OS06G0142650, OS05G0242000, OS01G0841650, OS05G0119200, OS02G0601300, OS01G0615850, OS02G0779300, OS02G0106700, OS07G0558000, OS06G0181800, OS01G0358100, OS10G035OS00, OS03G0439700, OS01G0532300, OS02G0188000, OS05G0479800, OS08G0189900, OS11G0705900, OS06G0535200, OS03G0333200, OS08G0122000, OS02G0477700, OS01G0176200, OS06G0603800, OS01G0315800, OS01G0451700, OS02GOS06600, OS02G0774500, OS01G0476700, OS12G0145200, OS01G0939500, OS05G0485300, OS01G0348000, OS05G0415800, OS10G0471400, OS05G0162200, OS02G0780000, OS12G0133400, OS01G0608200, OS03G0857900, OS06G0275600, OS01G0577500, OS01G0827500, OS03G0128500, OS01G0755100, OS01G0432100, OS02G0131200, OS01G0411800, OS03G0129800, OS06G0222100, OS01G0593200, OS09G0482900, OS06G0605600, OS05G0472400, OS11G0703400, OS05G01OS000, OS08G0152366, OS03G0377300, OS02G0192400, OS05G0357100, OS07G0693100, OS01G0574500, OS11G0184900, OS07G0593000, OS10G0365800, OS01G0865400, OS06G0230100, OS01G0536433, OS02GOS14712, OS01G0630900, OS10G0161400, OS06G0603300, OS09G0483800.

### Exemplary Exodermis-Enriched Promoters

Solyc12g005785, So1yc08g066890, So1yc07g049460, So1yc08g081555, So1yc09g082530, So1yc04g081860, So1yc07g052530, So1yc00g095860, So1yc08g078920, So1yc07g052540, Solyc10g076240, SolycO1g111230, So1yc08g075830, So1yc09g065430, So1yc12g006110, So1yc00g072400, So1yc10g076243, So1yc09g074890, So1yc07g047740, So1yc06g064960, So1yc05g012580, Solyc 10g037880, Solyc 12g096620, Solyc 11g072110, So1yc08g066930, SolycO1g081250, So1yc03g005760, So1yc02g084850, Solyc10g007930, So1yc05g046020, Solyc12g049680, So1yc02g070080, So1yc06g060620, So1yc08g081780, Solyc 12g011030, So1yc09g075670, Solyc 11g012360, So1yc07g049240, Solyc 10g009150, So1yc03g096420, So1yc08g074682, So1yc05g007470, Solyc12g097080, So1yc06g011350, So1yc08g014000, So1yc08g068780, So1yc09g082270, So1yc06g067870, So1yc08g061970, Solyc 11g066270, So1yc08g079190, So1yc07g055060, So1yc02g092670, So1yc03g115690, So1yc09g007770, Solyc10g085880, So1yc03g120475, So1yc02g065780, So1yc08g066880, So1yc01g090610, So1yc01g066910, So1yc01g108860, Solyc 10g083460, Solyc 11g031950, So1yc08g008OS0, So1yc04g007400, Solyc11g011190, So1yc02g080200, So1yc06g060760, So1yc04g077670, So1yc08g079200, So1yc06g066830, So1yc09g089830, So1yc04g007750, Solyc12g009650, So1yc09g072590, So1yc03g096030, So1yc06g073460, So1yc07g043130, So1yc02g089250, So1yc09g098620, So1yc09g007760, So1yc01g109500, Solyc 11g013810, So1yc06g060070, So1yc08g005960, So1yc06g075360, So1yc08g081190, SolycO1g096420, So1yc06g075650, Solyc12g005940, So1yc09g008320, Solyc12g056800, Solyc12g013690, So1yc02g086880, So1yc01g105410, So1yc09g014280, So1yc12g087940, So1yc03g111310, So1yc01g106780, So1yc01g097520, So1yc07g016215, So1yc02g080640, So1yc02g081400.

Promoter designations are from So1 Genomics Network database, genome version 513.0.

### Exemplary Fruit Epidermis-Enriched Promoters

Solyc03g116100, Solyc05g053550, Solyc02g083860, Solyc11g013110, Solyc05g052240, Solyc09g091510, Solyc10g083440, Solyc02g089770, Solyc10g075090, SolycO1g079620, Solyc09g042670, Solyc06g060570, Solyc09g090980, Solyc09g092270, Solyc07g049440, Solyc10g075070, Solyc03g115220

### Exemplary Phellem-Enriched Promoters

Solyc12g036480, Solyc02g084790, Solyc06g009010, Solyc06g074390, SolycO1g090460, Solyc09g008250, Solyc11g072600, Solyc05g055480, Solyc09g008030, Solyc07g063420

### Exemplary Endodermis-Enriched Promoters

Solyc01g016460, Solyc01g067180, Solyc01g067230, Solyc01g067610, Solyc01g080580, Solyc01g081177, Solyc01g086893, Solyc01g090840, Solyc01g102450, Solyc01g108OS0, Solyc02g068645, Solyc02g083790, Solyc02g084260, Solyc02g085285, Solyc02g088517, Solyc02g088600, Solyc02g088983, Solyc03g046207, Solyc04g008780, Solyc04g0S1427, Solyc05g005877, Solyc05g013207, Solyc06g043260, Solyc06g043275, Solyc06g054600, Solyc06g072650, Solyc07g018144, Solyc08g061107, Solyc08g065820, Solyc09g010564, Solyc09g037087, Solyc09g037125, Solyc09g037130, Solyc09g065490, Solyc10g008620, Solyc10g044543, Solyc10g047643, Solyc10g074680, Solyc11g012563, Solyc11g027920, Solyc 11g068630, Solyc 12g0OS040, Solyc 12g0OS130, Solyc 12g006225, Solyc 12g038350, Solyc12g042800, Solyc12g096270.

### Exemplary Drought-Inducible Promoters

Solyc06g076760, Solyc03g025810, Solyc12g010545, Solyc03g007230, Solyc12g006OS0, Solyc12g008430, Solyc02g086530, Solyc09g015070, Solyc12g089350, Solyc06g048860, Solyc06g068160, SolycO1g096320, Solyc11g071350, Solyc09g090790, Solyc09g082290, Solyc01 g100090, So1yc02g090210, So1yc05g053160, Solyc01 g060260, Solyc 10g008700, So1yc01g006620, Solyc04g011600, So1yc03g006360, Solyc03g117800, Solyc11g067190, SolycO1g109920, So1yc09g097760, So1yc06g060970, So1yc08g067260, So1yc05g010330, So1yc03g112590, So1yc06g067980, Solyc10g078770, SolycO1g057000, So1yc08g078550, Solyc01g111040, So1yc12g009680, So1yc03g097585, So1yc01g087180, So1yc09g082550, Solyc01 g103060, So1yc02g079640, So1yc07g055560, So1yc02g072540, Solyc11g009100, Solyc11g066700, So1yc08g079270, Solyc12g098900, So1yc06g076800, So1yc09g082340, So1yc06g060970, So1yc09g082280, So1yc03g097620, So1yc03g019820, SolycO1g099880, SolycO1g095320, So1yc09g015070, So1yc03g025810, So1yc06gOS1860, So1yc03g006360, Solyc11g007807, Solyc12g006OS0, Solyc12g098900, Solyc02g084850, Solyc02g061800, Solyc09g090800, Solyc10g079150, SolycO1g109920, Solyc03g044600, Solyc03g065250, Solyc08g081740, Solyc10g083690, Solyc03g097600, Solyc06g069070, Solyc04g071770, SolycO1g095305, SolycO1g096320, Solyc08g062960, Solyc03g095650, Solyc09g082300, Solyc03g007790, Solyc03g096670, Solyc08g078757, Solyc03g007230, Solyc03g013440, Solyc06gOS0800, Solyc08g075150, Solyc 10g008700, Solyc04g016430, Solyc04g007470, Solyc10g024490, Solyc06g076400, Solyc09g083OS0, Solyc01 g109810, SolycO1g057000, Solyc06g008580, Solyc08g068150, Solyc09g005610, Solyc12g010545, Solyc04g072700.

Solyc06g009370 is a robust meristematic cortex enriched promoter, stress independent in lateral roots (independent from drought and waterlogging in meristematic cortex and mature cortex).

Solyc08g081150 is a robust meristematic cortex enriched promoter, stress independent in lateral roots (independent from drought and waterlogging in meristematic cortex and mature cortex.

Additional exemplary endodermis enriched promoters:
Solyc01g016460, Solyc01g067180, Solyc01g067230, Solyc01g067610, Solyc01g080580, Solyc01g081177, Solyc01g086893, Solyc01g090840, Solyc01g102450, Solyc01g108OS0, Solyc02g068645, Solyc02g083790, Solyc02g084260, Solyc02g085285, Solyc02g088517, Solyc02g088600, Solyc02g088983, Solyc03g046207, Solyc04g008780, Solyc04gOS1427, Solyc05g005877, Solyc05g013207, Solyc06g043260, Solyc06g043275, Solyc06g054600, Solyc06g072650, Solyc07g018144, Solyc08g061107, Solyc08g065820, Solyc09g010564, Solyc09g037087, Solyc09g037125, Solyc09g037130, Solyc09g065490, Solyc10g008620, Solyc10g044543, Solyc10g047643, Solyc10g074680, Solyc11g012563, Solyc11g027920, So1yc11g068630, So1yc12g0OS040, So1yc12g0OS130, So1yc12g006225, So1yc12g038350, So1yc12g042800, So1yc12g096270.

Also provided are methods of suppressing expression or activity of a polypeptide substantially identical to a protein of Table 1 or any one of SEQ ID NOS: 1-20 in a plant using an upstream open reading frame (uORF) to reduce expression at the level of mRNA translation.

Methods for transformation of plant cells are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens or Rhizobium rhizogenes-mediated transformation. Transformation means introducing a nucleotide sequence in a plant in a manner to cause stable or transient expression of the sequence.

In some embodiments, in planta transformation techniques (e.g., vacuum-infiltration, floral spraying or floral dip procedures) are used to introduce the expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et al. 1993 C. R. Acad. Sci. 316:1194-1199; Chung et al. 2000 Transgenic Res. 9:471-476; Clough et al. 1998 Plant 116:735-743; and Desfeux et al. 2000 Plant Physiol 123:895-904). In these embodiments, seed produced by the plant comprise the expression cassettes encoding the proteins. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

An expression cassette can be integrated into the genome of the plant cells, in which case subsequent generations will express the encoded proteins. Alternatively, the expression cassette is not integrated into the genome of the plants cell, in which case the encoded protein is transiently expressed in the transformed cells and is not expressed in subsequent generations.

Any plant can be modified as described herein to have modulated amounts of suberin. Exemplary plants include species from the genera Arachis, Asparagus, Atropa, Aven, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malta, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. In some embodiments, the plant is a graminaceous or solanaceous plant. Exemplary graminaceous plants include but are not limited to rice, maize, wheat, sorghum, barley, rye, oat, millet, fescue, bluegrass, and ryegrass.

### EXAMPLES

### Example 1. Materials and Methods

Data Availability

Rice translatome abundance data can be viewed for individual genes and groups of genes for the cell population and environmental condition comparisons in a spatialHeatmap at the baileyserreslab.org/website. ATAC-seq and RNA-seq tracks can be viewed in a JBrowse instance in the website. The accession number for the raw TRAP-seq and genomic DNA-based ATAC-seq data reported in this paper is at NCBI GEO as GSE180100.

Experimental Model and Subject Details

Rice Genetic Material

Rice (Oryza sativa cv. japonica, Nipponbare) was used. Previously reported INTACT destination vectors (Reynoso et al., 2018) were used to construct p35S:INTACT BLRP-eGFP-OsWIP2/pH7WG-OsNTF2, as well as pCMZ:INTACT and pQHB:INTACT OsWPP-GFP-BLRP/pH7WG-OsNTF1. Promoter:TRAP constructs were prepared with the TRAP destination vector pH7WG-OsTRAP that was generated by modification of p35S:HF-OsRPL18 (Zhao et al., 2017). In this vector, the Gateway recombination site for promoter insertion is followed by a chimeric ribosomal protein consisting of a 6xHis-FLAG-3xGly tag, GFP and OsRPL18/eL18 (OS03G0341100). The promoters were recombined into pENTR-D/TOPO (Invitrogen) and introduced into the INTACT and TRAP vectors by use of LR Clonase II Enzyme mix (Invitrogen). Constructs were confirmed by Sanger sequencing. To enable promoter domain expression analysis by β-glucuronidase (GUS) staining of tissue, promoters were cloned upstream of the uidA gene of E. coli using the vector pHGWF S7 (https://gateway.psb.ugent.be/search). T-DNA plasmids were transformed into O. sativa japonica cv. Nipponbare embryogenic calli, derived from mature seed embryos, by use of Agrobacterium tumefaciens (EHA105A) as described by (Reynoso et al., 2018; Sallaud et al., 2003) or at the UC Davis Plant Transformation Facility. Hygromycin-resistant regenerated plantlets were transferred to pots filled with Profile® Greens Grade™ in a greenhouse and grown (28° C. day/20° C. night under natural light conditions) at the University of California, Riverside and allowed to self-pollinate. Regional and cell population specificity of promoters regulating the GFP-containing INTACT and TRAP transgenes were imaged by laser scanning microscopy using a confocal Leica SP5 with a 488 nm excitation laser at 50% power, a 56.7 μm pinhole, and 650-1000 smart gain. Alternatively, a Zeiss 880 Inverted with Airyscan was used with 488 and 561 excitation laser in Lambda mode for linear unmixing to deconvolute GFP signal from autofluorescence. Brightfield images were obtained to visualize root anatomy. Promoters regulating uidA were evaluated by GUS staining of tissue or tissue sections and subsequent brightfield imaging. Promoter evaluation was performed on seedlings grown on sterile agar and in pots cultivated in the greenhouse as described below.

Rice Growth Conditions and Stress Treatments

For plants used for bulking of seed or stress treatments in the greenhouse, seeds were imbibed for 2 d in 5% (v/v) Liquid Smoke™ (Colgin) at 28° C. in darkness to promote consistent germination. The seeds were then rinsed with distilled water and returned to 28° C. in darkness for 24 h. Twenty one germinated seeds were transferred to pots (15 cm diameter and 18 cm height), filled with Profile® Greens Grade™ and placed in a greenhouse at the University of California, Riverside California during June and July 2016. The temperature was controlled at 28° C. for 16 h per day and at 25° C. for 8 h per night. Pots were placed in trays and watered daily with fresh water (depth of 1 cm water tray). After 10 d of growth, irrigation was switched to fertilizer water (Peters® Excel: 21-5-20 at 100 ppm N). Each pot contained 21 plants (combined as a single replicate). The location of each pot was randomized in multiple trays/tanks. After 21 d of development (from imbibition), when plants were at the V4 to V5 growth stage, four water regimes were applied. For water-sufficiency (control), pots were maintained by daily irrigation with fertilized water. For water deficit, pots were removed from the tray and watering was stopped. For waterlogging, pots were placed in tanks (56-qt clear storage box; Sterilite® 1659) and flooded with water to 2-3 cm above the root/shoot junction. For submergence, pots were transferred to tanks (grey plastic Rubbermaid® FG265500 containers) at solar midday and fully submerged with water, with the flood line 70 cm above the root/shoot junction. The containers were covered with two layers of shade cloth (SHANS 90% UV Block). A fiber optic oxygen meter (Neofox Sport, Ocean Optics, Dunedin, USA) was used to determine the percentage of dissolved oxygen in the submergence tanks in four replicates, measured at the start and end of the experiment. Oxygen content was stable at 18.90±0.03% at the start (0 d) and end of the experiment. The submergence experiment was completed independently one week following the water deficit and waterlogging experiments and therefore has its own independent control condition, totaling ten environments profiled. Five days after treatments commenced, control, submerged, or waterlogged plants were harvested or returned to normal watering conditions at midday for 24 h before harvesting (recovery). Harvesting began one hour before subjective noon and took four to five hours for a team to complete sampling.

Plants exposed to water deficit were harvested on day five or six, depending on the leaf relative water content (RWC) determined the prior day for control and stressed plants that were not harvested for tissue. Leaf three was removed at the base of the plant and fresh, turgid, and dry weights were determined (Fukao et al., 2011). RWC was calculated as [(fresh weight−dry weight)/(turgid weight−dry weight)]×100. RWC was also assessed for harvested control and water deficit stress plants on the day of harvest. On day four at midday, leaf RWC of water-deprived plants reached 72.9±4.2%. On day five (at harvest) the RWC of well-watered plants was 95.2±3.9%. Total shoot elongation and root biomass were determined. Biological replicates were produced on separate consecutive days. Whole root systems were harvested in five independent replicates by rapid immersion in liquid nitrogen and kept at −80° C. until processing.

For the field study, seedlings were germinated in Magenta boxes on selective medium after sterilization, transferred to Profile®, and grown for twelve days in the greenhouse before transplantation into a paddy maintained at eight to ten cm depth for 35 days. Planting of genotypes was based on a randomized design with ten plants per biological replicate per genotype. The genotype of each plant was confirmed after planting. The Agricultural Operations field at the University of California, Riverside Agricultural Experiment Station was prepared and pre-fertilized. Soil samples taken a week before harvesting in the wet paddy had elemental contents of 52.27±16.77 ppm NO3-N, 14.00±0.32 Olsen-P and 97.00±5.10 X-K.

For growth on agar plates, the hull was removed, and seeds were surface sterilized in 50% (v/v) bleach for 30 min, rinsed in sterile distilled water and arranged on plates (10 cm×10 cm) containing 0.5× (w/v) Murashige and Skoog standard medium (MS), 1% (w/v) agar and 1% (w/v) sucrose. Plates were placed in a chamber with a 16 h day [110 μEm⁻² s⁻¹]/8 h night light cycle at 28° C./25° C. day/night for seven days. Whole root systems were harvested into liquid nitrogen for five independent biological replicates. All lines used to evaluate each condition were tested in the same experiment.

Method Details

Nuclei purification by INTACT for ATAC-seq

Nuclei were purified from frozen and pulverized tissue as described previously for rice by (Reynoso et al., 2019) according to the methods of (Deal, R. B., and Henikoff, S. (2010). A simple method for gene expression and chromatin profiling of individual cell types within a tissue. Dev. Cell 18, 1030-1040; Maher, K. A., Bajic, M., Kajala, K., Reynoso, M., Pauluzzi, G., West, D. A., Zumstein, K., Woodhouse, M., Bubb, K., Dorrity, M. W., et al. (2018). Profiling of Accessible Chromatin Regions across Multiple Plant Species and Cell Types Reveals Common Gene Regulatory Principles and New Control Modules. Plant Cell 30, 15-36.). To reduce cellular debris, the 70 μm filtration step was replaced by a 30 μm filtration step (Celltrics) and performing the centrifugation at 1000×g at 4° C. for 15 min. Nuclei were counted prior to ATAC-seq for all lines except pQHB:INTACT, due to low yield. ATAC-seq was performed by Tn5 insertion into chromatin of purified nuclei using the Nextera DNA Library Preparation Kit on nuclei as described (Reynoso et al., 2019). ATAC-seq libraries were sequenced on the NextSeq 500 at the UC Davis DNA Technologies Core to obtain 36 nt paired-end reads.

Polysomal mRNA Purification by TRAP and Total RNA Isolation and RNA-Seq Library Construction

TRAP was performed as previously described (Mustroph, A., Juntawong, P., and Bailey-Serres, J. (2009b). Isolation of plant polysomal mRNA by differential centrifugation and ribosome immunopurification methods. Methods Mol. Biol. 553, 109-126; Reynoso et al., 2015) with slight modifications through polyA RNA purification as described by (Reynoso et al., 2019). Isolation of total RNA and the synthesis of non-directional mRNA sequencing libraries by random priming (Townsley, B. T., Covington, M. F., Ichihashi, Y., Zumstein, K., and Sinha, N. R. (2015). BrAD-seq: Breath Adapter Directional sequencing: a streamlined, ultra-simple and fast library preparation protocol for strand specific mRNA library construction. Front. Plant Sci. 6.) also followed prior methods (Reynoso et al., 2019). Samples included at least three biological replicates for each condition and line. Sequencing was performed with a Illumina HiSeq 3000 to obtain 50 nt single-end reads.

EdU Staining of Root Tips and Root Length Measurements

Seeds with seed coats removed were sterilized in 50% (v/v) bleach for 10 min followed by rinsing with ddH2O. All growth was under a 16 h light (115 μEm⁻² s⁻¹)/8 h dark cycle at 22° C. in a growth chamber. For submergence, each seed was planted at the top of 6 mL of moist Profile® in a 23 mL borosilicate glass tube (16 mm×150 mm; Fisher), grown for 7 d before full submergence in ddH2O at Zeitgeber time 6, for 24 h in darkness. For recovery, tubes were drained and returned to the chamber for 24 h. Primary roots were evaluated, as seedling crown roots were more variable in length and number. For primary root length measurements, seedlings of similar coleoptile length were fully submerged in darkness for 3 d or maintained in air under darkness for 3 d. For water deficit, seedlings were grown in pots (6 cm diameter×6 cm height) filled with Profile®. On day 8, 50-75% of the endosperm was gently excised and plants were deprived of water for 3 d at which time leaves were rolled. Roots were sampled or rewatered and then sampled after 24 hours of recovery, with corresponding 11 d old controls for EdU staining or 12 d old controls for primary root length. Root tips were stained using the Click-iT® EdU Imaging Kit (Invitrogen) according to the manufacturer's protocol by immersion of the apical region of primary root in 1001 μM EdU (5-ethynyl-2′-deoxyuridine) for 1 h and washed twice with 100 mM Tris-HCl (pH 8.0) before placement on a 1 mm thick glass microscope slide with a high precision coverslip (170±5 μM thickness, Thor Labs, #1.5H) and incubation in the Click-iT EdU detection cocktail for 30 min in darkness. Root sections were washed with 100 mM Tris-HCl (pH 8.0) and immersed in 0.4 μg/μL DAPI (4′,6-diamidino-2-phenylindole) for 5 min and washed again before imaging with a Zeiss LSM 880 upright microscope. Acquisition of DAPI and EdU fluorescence was with an excitation laser of 395 and 495 nm, and gain of 544 and 800, respectively. Fluorescent signal was quantified for the elongation zone to the root apex using the combined Z stack image.

Suberin Staining

Suberin was visualized after Fluorol Yellow (FY) staining. Briefly, roots were incubated in 0.01% (w/v) FY solution in lactic acid (Sigma) for 60 min, rinsed three times with distilled water, and counterstained with 0.5% (w/v) aniline blue for 5 min under vacuum, and then 25 min at room temperature in darkness. Roots were observed by Confocal Laser Scanning microscopy in a Leica SP5 using GFP settings, smart gain (800), laser at 50% and pinhole set at Airy 1. Corrected Total Cell Fluorescence (CTCF) was calculated using ImageJ as described (Clark et al., 2019). Briefly, integrated density of fluorescence is divided by the area of the cells multiplied by the mean fluorescence of a region with no signal defined as background.

Perls-DAB Staining

Plants were grown in three replicates staggered by one day in a greenhouse in Riverside, CA, as described above, with five plants per pot. Water was withheld for 29 days to achieve similar dehydration levels (onset of leaf rolling) to the TRAP-seq experiments; a longer duration was required due to shorter day length and cloudy weather. Whole root systems of plants (stage V5-6) were harvested into fixation solution (MeOH, CHC13, HOAc; 6:3:1) and Fe was visualized using Perls staining followed by diaminobenzidine (DAB) intensification (Brumbarova and Ivanov, 2014). Photographs of whole root systems (n=9 for WD, n=8 for CON, 2-3 per replicate) were scored from 1 (least staining intensity) to 4 (most staining intensity) by five researchers who were blinded to the treatment and the experimental design. Crown roots (2-8 cm) were sectioned approximately 1-2 cm from the root tip by embedding in 4% (w/v) agarose and generating 100 μm sections using a EMS5000 Oscillating Tissue Slicer. Sections were imaged using the brightfield setting on a Keyence BZ-X710 Microscope with 20× magnification.

Quantification and Statistical Analyses

ATAC-Seq and RNA-Seq Data Processing

Short read processing, quality assessment, alignment to the genome were performed as described (Reynoso et al., 2019) using the University of California, Riverside Institute for Integrative Genome Biology high performance bioinformatics cluster (http://www.bioinformatics.ucr.edu/), supported by NSF MM DBI 1429826 and NIH S10-OD016290. For these analyses, we used R packages from Bioconductor including systemPipeR (H Backman and Girke,2016) and IGRSP1.0-30 genome (http://plants.ensembl.org/Oryza_sativa/Info/Index) with the Bowtie2/Tophat2 suite. Read count data for features of exons-by-genes were obtained with the summarizeOverlaps function of GenomicRanges (Juntawong, P., Girke, T., Bazin, J., and Bailey-Serres, J. (2014). Translational dynamics revealed by genome-wide profiling of ribosome footprints in Arabidopsis. Proc. Natl. Acad. Sci. U.S.A 111, E203—E212). For visualization of data in a genome browser, Samtools 1.0.9 was used to convert reads from SAM to sorted BAM files. ATAC Bowtie2 aligned SAM files were filtered to retain only reads with a mapping quality score of 10 or higher. BedTools 2.26 genomeCoverageBed was used to make bigwig files which were converted to UCSC bedGraphtoBigWig 332-0 files with rpkm normalization.

RNA-Seq Differential Expression, Mapping of Chromatin Accessibility, Identification of Transposase Hypersensitive Sites, and Evaluation of Accessibility Changes Between Conditions

A pipeline in R that we developed previously (Reynoso et al., 2019) was used to obtain different contrasts in mRNA abundance based on environment, including log 2 Fold Change (FC) values and adjusted P values (adj.P.Val). This included use of the limma-voom Bioconductor package in R for quantile normalization and FC calculations (Ritchie et al., 2015) and production of multidimensional scaling (MDS) plots using Glimma (Su, S., Law, C. W., Ah-Cann, C., Asselin-Labat, M. -L., Blewitt, M. E., and Ritchie, M. E. (2017). Glimma: interactive graphics for gene expression analysis. Bioinformatics 33,2OS0-2052.) using genes with more than 3 count per million in at least three biological replicates. The total number of detectable mRNAs for any given promoter: TRAP translatome was consistent across treatments, indicating translatome differences are not due to differences in library complexity. The voom function on the limma package was used to calculate normalized counts. Following normalization, count data were used to calculate transcripts per million reads (TPM). Top varying genes were analyzed for dominant patterns of coregulation as described previously. Evaluation of enrichment in Gene Ontology terms was performed with systemPipeR (H Backman, T. W., and Girke, T. (2016). systemPipeR: NGS workflow and report generation environment. BMC Bioinformatics 17, 388) using rice GO definitions obtained from BioMart. To integrate p35S polyA and TRAP stress changes for roots, limma voom log 2 FC values for DEGs in each condition (|log2 FC|>1 and Padj<0.01]) compared to control were clustered using the Partitioning Around Medoids (PAM) method with k=15 clusters using gplots, cluster, e1071, and RcolorBrewer R packages. Several k values were tested for resolution of correlated genes. p35S ATAC log 2 FC values were calculated for normalized reads located up to 2 kb upstream of the ATG for genes in each cluster and median value plotted in the heatmap. Peaks in ATAC data were found running the HOMER package function “Findpeaks” with the parameters “-minDist 150” “-region” and “-regionRes 1”. THSs locations were kept if they overlapped at least by 50 bp in biological replicates of the same condition. This was done using the “findOverlapsOfPeaks” function of the ChIPPeakAnno package (Zhu, L. J., Gazin, C., Lawson, N. D., Pages, H., Lin, S. M., Lapointe, D. S., and Green, M. R. (2010). ChIPpeakAnno: a Bioconductor package to annotate ChIP-seq and ChIP-chip data. BMC Bioinformatics 11, 237). Reproducible peaks are referred to as THSs.

The fraction of reads in peaks was calculated using the frip( )function of the encodeChIPqc package. Read alignments overlapping with THSs coordinates were quantified in three biological replicates for each sample. Counts for each THSs were statistically evaluated using limma voom after quantile normalization. THSs with a log 2 fold change value of more than 1, or less than −1, and adjusted p-value<0.05 were identified as dTHSs, which refer to chromatin regions where chromatin is more accessible or less accessible between two conditions.

We developed a spatialHeatmap Shiny App instance to visualize the cell and tissue level gene expression data of this study interactively within the context of anatomical images from rice roots (https://github com/jianhaizhang/spatialHeatmap) (Zhang J, Hayes J, Zhang L, Yang B, Frommer W, Bailey-Serres J, Girke T (2020). spatialHeatmap: spatialHeatmap. R package version 1.2.0, https://github.com/jianhaizhang/spatialHeatmap.).

Network Construction

Coexpression networks were generated using all rice RNA-seq (TRAP and Total) data from this work and (Kajala, K., Gouran, M., Shaar-Moshe, L., Mason, G. A., Rodriguez-Medina, J., Kawa, D., Pauluzzi, G., Reynoso, M., Canto-Pastor, A., Manzano, C., et al. (2021). Innovation, conservation, and repurposing of gene function in root cell type development. Cell.) as described in (Wisecaver, J. H., Borowsky, A. T., Tzin, V., Jander, G., Kliebenstein, D. J., and Rokas, A. (2017). A Global Coexpression Network Approach for Connecting Genes to Specialized Metabolic Pathways in Plants. Plant Cell 29, 944-959) using scripts from https://github.itap.purdue.edu/jwisecav/mr2mods. Briefly, the Pearson correlation coefficient (PCC) is calculated for all possible pairs of genes based on normalized counts across all the data provided. These are used to calculate the mutual rank score (the geometric mean of the reciprocal ranks) for each pair of genes. Mutual rank scores are converted to network edge weights with five different exponential decay functions, producing five different networks with varying stringency for connections. Finally, ClusterONE is used to call modules for each of these networks (Nepusz, T., Yu, H., and Paccanaro, A. (2012). Detecting overlapping protein complexes in protein-protein interaction networks. Nat. Methods 9, 471-472).

To model gene regulatory networks and infer hierarchies, Taiji version 0.5.0 (Zhang, K., Wang, M., Zhao, Y., and Wang, W. (2019a). Taiji: System-level identification of key transcription factors reveals transcriptional waves in mouse embryonic development. Sci Adv 5, eaav3262) was run on fifteen paired ATAC-seq and TRAP-seq datasets, using a.meme file generated from the CisBP database (Lambert, S. A., Yang, A. W. H., Sasse, A., Cowley, G., Albu, M., Caddick, M. X., Morris, Q. D., Weirauch, M. T., and Hughes, T. R. (2019). Similarity regression predicts evolution of transcription factor sequence specificity. Nat. Genet. 51, 981-989), with the addition of the hypoxia-responsive promoter element (HRPE) motif reported previously (Gasch, P., Fundinger, M., Müller, J. T., Lee, T., Bailey-Serres, J., and Mustroph, A. (2016). Redundant ERF-VII Transcription Factors Bind to an Evolutionarily Conserved cis-Motif to Regulate Hypoxia-Responsive Gene Expression in Arabidopsis. Plant Cell 28, 160-180) assigned to OsERF66 and 67. To identify genes with specific activity (PageRank scores uniquely elevated in a certain cell population or condition), the output gene rank matrix was filtered to TFs with a row Z-score of at least 2.5 in at least one condition/cell population, and then clustered with partitioning around medoids (PAM) clustering in R (Maechler, M (2021). cluster: “Finding Groups in Data”: Cluster Analysis Extended Rousseeuw et al. Meguro, A., and Sato, Y. (2014). Salicylic acid antagonizes abscisic acid inhibition of shoot growth and cell cycle progression in rice. Sci. Rep. 4, 4555). cluster: “Finding Groups in Data”: Cluster Analysis Extended Rousseeuw et al. Meguro, A., and Sato, Y. (2014). Salicylic acid antagonizes abscisic acid inhibition of shoot growth and cell cycle progression in rice. Sci. Rep. 4, 4555). These clusters were summarized using word clouds (Fellows, I. (2012). wordcloud: Word clouds. R Package Version 2, 109). wordcloud: Word clouds. R Package Version 2, 109) based on the frequency of TF Families in each cluster. To visualize networks, the network output from Taiji (Zhang, K., Wang, M., Zhao, Y., and Wang, W. (2019a). Taiji: System-level identification of key transcription factors reveals transcriptional waves in mouse embryonic development. Sci Adv 5, eaav3262) was filtered to only edges where genes in the cluster are both a source and target node, then loaded into Cytoscape (Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res. 13, 2498-2504), laid out with “Hierarchical layout,” and manually adjusted for spacing. Node size was assigned by PageRank score and color by TPM.

To develop the suberin network, after testing enrichment of TFBSs in their proximal THS for the coexpression module, we searched the accessible regions 2 kb upstream or 1 kb downstream of the transcriptions start sites (TSSs) of module genes of enriched TFBSs that were associated with a TF that was expressed at least 10 TPM in any of the LSI1 greenhouse TRAP conditions. Additionally, we also searched for TFBSs associated with any TF Gene that was present in the coexpression module, regardless of whether or not it was enriched.

Source and target nodes were imported into Cytoscape (Shannon et al., 2003) and the NetworkAnalyzer tool in Cytoscape was used to calculate edge betweenness, and the “Edge-weighted spring embedded layout” was executed using edge betweenness.

Identification of Cell Population Enriched Genes (CPEGs)

ROKU (Kadota, K., Ye, J., Nakai, Y., Terada, T., and Shimizu, K. (2006). ROKU: a novel method for identification of tissue-specific genes. BMC Bioinformatics 7, 294) was run on mean TPM values for TRAP-seq data for each promoter: TRAP line for which data were generated in the field, with parameters set so that a gene could only be enriched in one cell population (upper.limit=0.2). These gene sets of CPEGs were tested for GO enrichment as previously described. This process was repeated independently for each condition/environment where at least three promoter: TRAP lines were assayed, and then these gene sets were intersected to identify environmentally conserved CPEGs for promoter: TRAP lines represented in at least two of three: plate, greenhouse, field experiments (pRSS1,pLSI1, pQHB, and p35S). To generate networks, as described for cell cycle and suberin above, THSs proximal to CPEGs for each class were input into AME to test for enrichment of the CisBP motifs, with all other THSs as a negative control. Enriched motifs that were associated with TF genes that themselves were CPEGs were searched using FIMO in THSs from 2 kb upstream to 1 kb downstream of CPEG genes, and these FIMO searches were used to generate networks in Cytoscape, with “Edge-weighted spring embedded layout” based on Edge betweenness.

Phylogeny

Gene orthology relationships for E2F/DP genes were determined following (Rokas, 2011). This was generated by downloading a hmm for the E2F TDP family from Pfam (http://pfam.xfam.org/family/PF02319), then searching the proteomes of Oryza sativa, Arabidopsis thaliana, Medicago truncatula, Solanum lycopersicum, Chlamydomonas reinhardtii, Vitis vinifera, Selaginella moellendorffii, Homo sapiens, and Sorghum bicolor for matching proteins using hmmsearch from hmmer3.3.2 (Finn, R. D., Clements, J., and Eddy, S. R. (2011). HM1ViER web server: interactive sequence similarity searching. Nucleic Acids Res. 39, W29—W37). Next, sequences were aligned with mafft 7.471 (Katoh and Standley, 2013) using default settings, then alignment trimmed with trimal 1.4.1 (Capella-Gutierrez, S., Silla-Martinez, J. M., and Gabaldon, T. (2009). trimAl: a tool for automated alignment trimming in large-scale phylogenetic analyses. Bioinformatics 25, 1972-1973) with −gt 0.5. Finally, a tree was generated with RAxML 8.2.12 (Stamatakis, A. (2014). RAxML version 8: a tool for phylogenetic analysis and post-analysis of large phylogenies. Bioinformatics 30, 1312-1313) with 100 bootstraps and −mPROTGAMMAAUTO. The resulting tree was midpoint rooted and visualized in FigTree 1.4.3 (Rambaut and Drummond).

### Example 2. Promoter Accessibility Regulation by Environmental Stimuli is Reversible and Highly Correlated with Transcript Dynamics

The extent to which the growth environment influences chromatin accessibility of conditionally expressed genes is largely unknown. To gain a broad perspective on environmentally influenced gene regulation, we assayed cell populations of root systems of plants in nine conditions: grown on sterile media in plates, cultivated in a paddy field, or established in pots in the greenhouse and then exposed to sublethal water extremes (FIG. 1A. The environmental conditions included well-watered drained control (CON), waterlogging (WL), complete submergence (SUB), or water deficit (WD)). The reversibility of each response was monitored by sampling after one day of recovery: rewatering after water deficit (WD+R), drainage after waterlogging (WL+R), and desubmergence (SUB+R). To establish an initial view of epigenome and translatome response signatures, we started with a p35S:INTACT line to monitor accessible chromatin by INTACT-ATAC-seq and a p35S:TRAP line to monitor the transcriptome and translatome and progressed to promoters that mark specific cell populations. The domains of expression of these and other promoters were visualized by GFP or GUS detection. As these promoters and ribosome synthesis may be conditionally regulated, we refer to each translatome monitored by the promoter name. Finally, we integrated the accessible chromatin and gene activity information by testing the enrichment of TF binding sites and inferring gene regulatory networks (FIG. 1B).

ATAC-seq reads from rice roots predominantly map within 2 kb upstream of the transcriptional start site of protein coding genes with high reproducibility and can be used to define regions of chromatin accessibility: transposase hypersensitive sites (THSs) that are differentially regulated (dTHSs). Water deficit and submergence, but not waterlogging, significantly enhanced or repressed regions of chromatin accessibility (FIG. 1C). These dTHSs generally reverse to pre-stress accessibility levels within 24 hours of rewatering or desubmergence. We found that chromatin accessibility is highly similar in roots grown under well-watered or waterlogging conditions, whereas older field grown plants have more closed than open chromatin regions compared to the well-watered greenhouse roots (FIG. 1C). Similar to the chromatin dynamics, the growth environment distinctly influences the transcriptome and translatome and these alterations in response to the sub-lethal water extremes are overall reversible within 24 hours of recovery. To assess whether environmental dynamics in chromatin accessibility are predictive of change in mRNA accumulation (transcriptome or translatome), we assigned each THS to the nearest gene in the genome. This confirmed that the genes proximal to conditionally open THS (up-dTHS) were significantly more upregulated and genes proximal to down-dTHS were significantly more downregulated in response to water extremes compared to the control (FIG. 1D). This trend holds for distal elements: dTHSs that are 2-12 kb from the nearest gene. Our data also illustrate that changes in promoter chromatin accessibility coordinate with changes in steady-state and ribosome-bound mRNA abundance for water deficit and submergence (FIG. 1E, as illustrated for genes induced by water deficit (cluster 1; includes ABA response and carbohydrate transport genes) and submergence (cluster 10; includes carbohydrate catabolism genes). The transcriptome and translatome profiles of plate-grown seedlings are distinct, largely due to light-responsive photosynthetic gene activity (cluster 5). Genome browser views illustrate epigenetic and transcript regulation of signature conditionally expressed genes (FIG. 1F). As dynamics in chromatin accessibility and translated mRNA are coordinated for most clusters of co-regulated genes (conditional AATAC vs. ATRAP correlation: WD, R 2=0.91 and SUB, R 2=0.94), we proceeded to combine THS and translatome data to elucidate conditional gene regulatory dynamics of distinct cell populations.

### Example 3. Water Deficit Translatomes Highlight Core Physiological Responses and Exodermal Plasticity in Suberin Synthesis

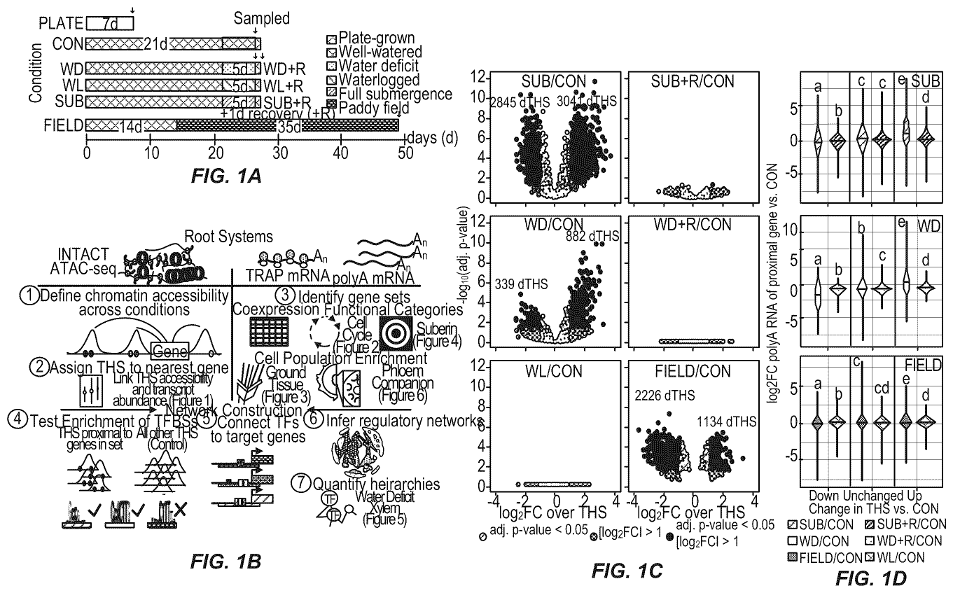

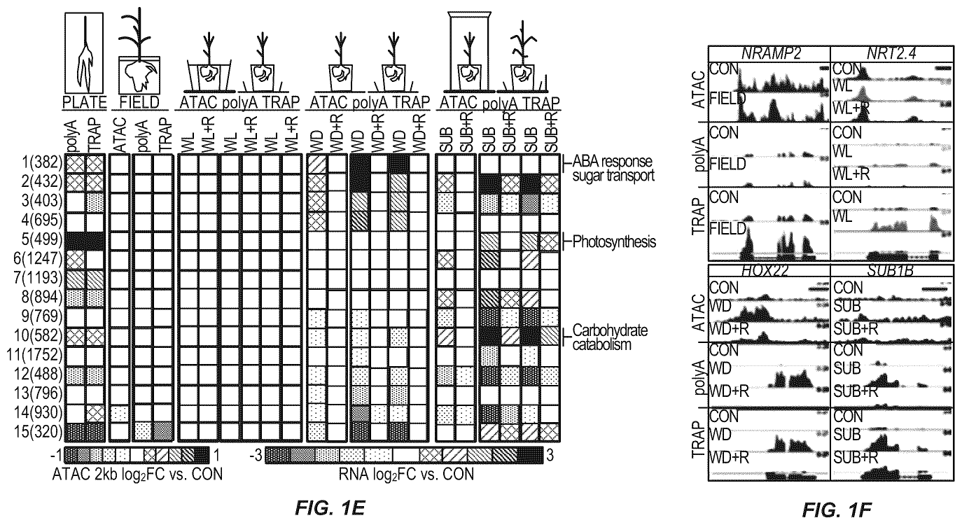

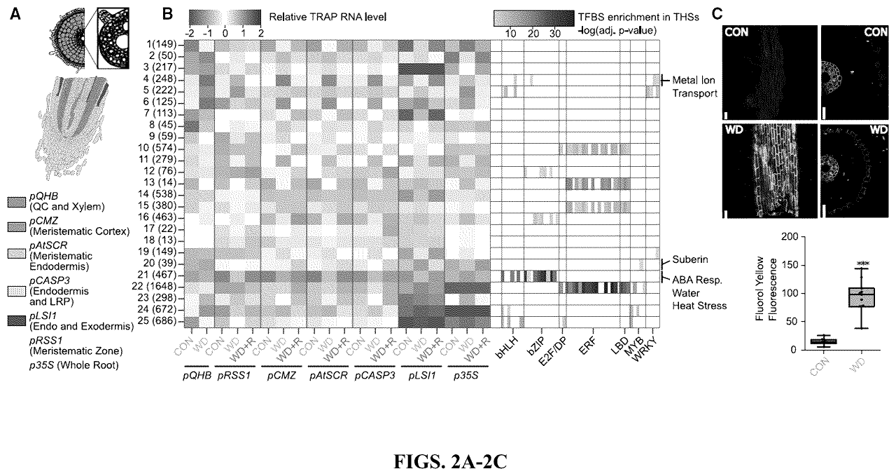

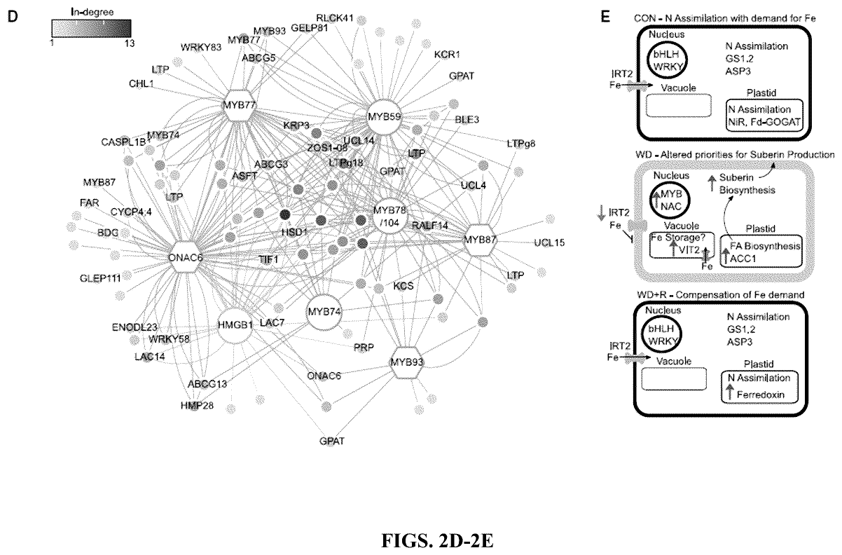

We found that water deficit invokes a strong ABA transcriptional response (Pattern 21, padj E-7) and distinct cell population-specific responses (FIGS. 2A & B). The promoters of Pattern 21 genes are conditionally accessible and enriched for bZIP TFBSs. These include the ABA Response Element (ABRE) bound by bZIP TFs that activate ABA responsive genes. A pronounced upregulated pattern specific to pLSI1-marked cells (Pattern 20) is enriched for genes involved in suberin biosynthesis (padj E-3). Fluorol Yellow staining confirms increased suberin deposition primarily in the exodermal cell layer under water deficit, whereas the endodermis is suberized independent of condition (FIG. 2C). With this observation, we hypothesize that these dynamics originate mainly in the exodermis and not the endodermis.

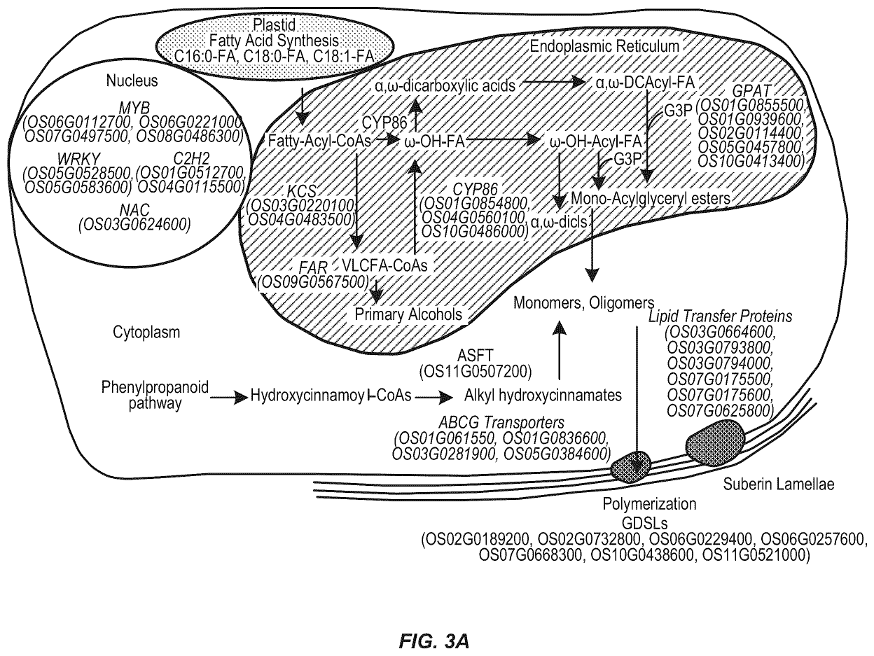

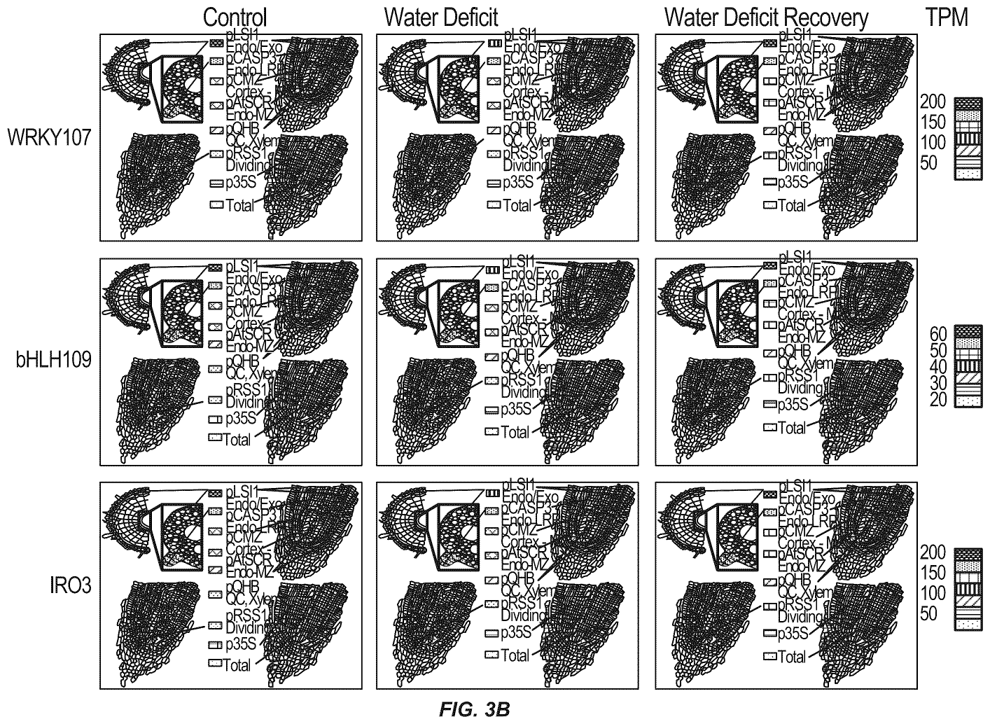

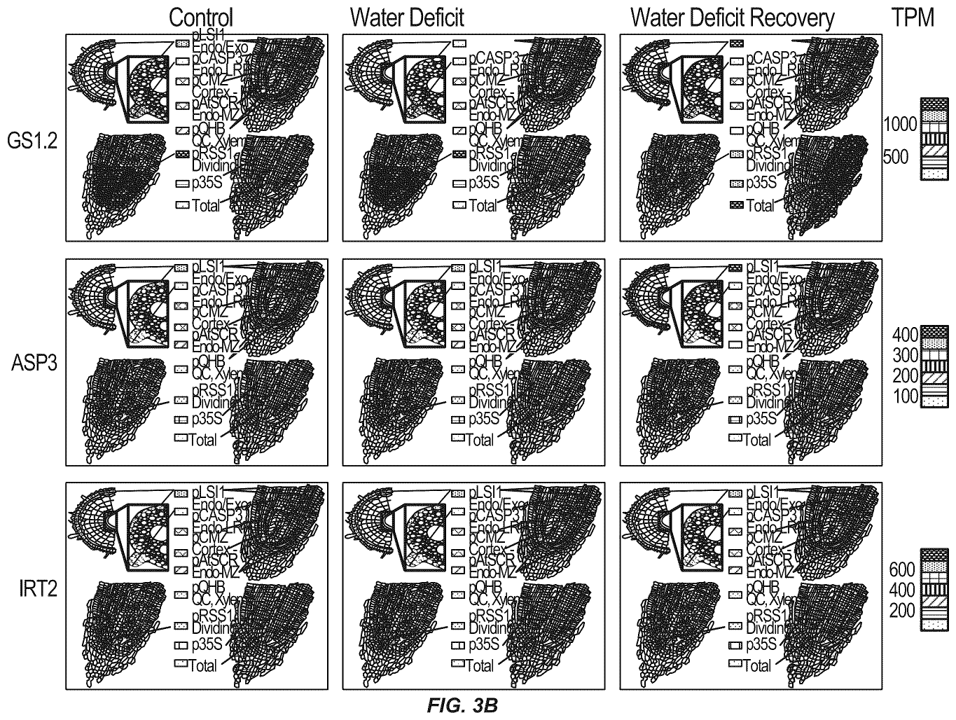

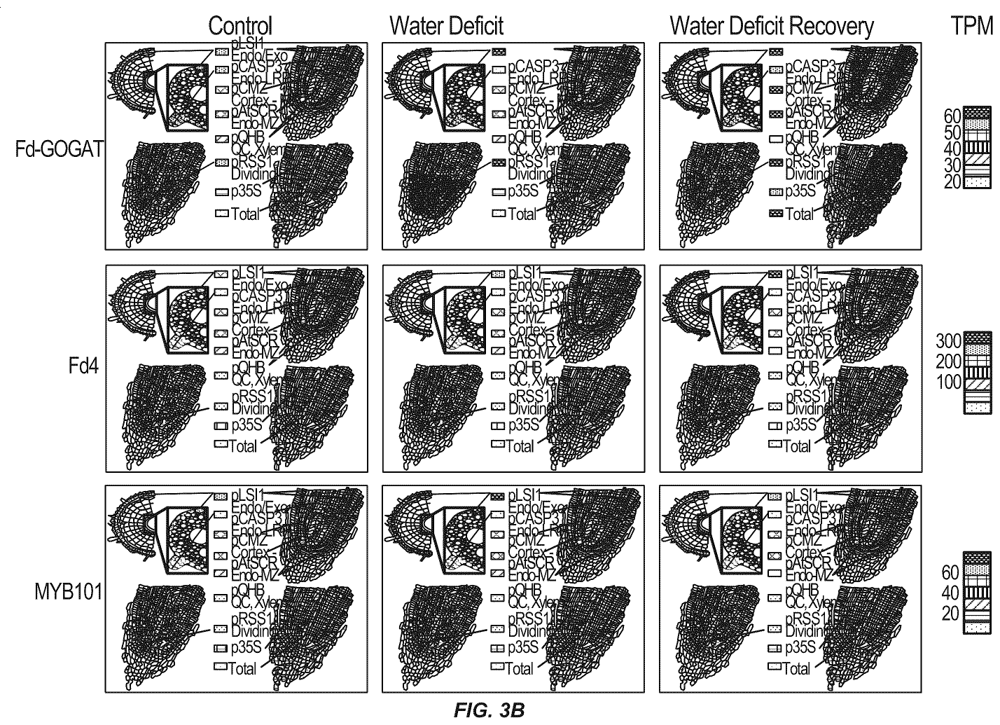

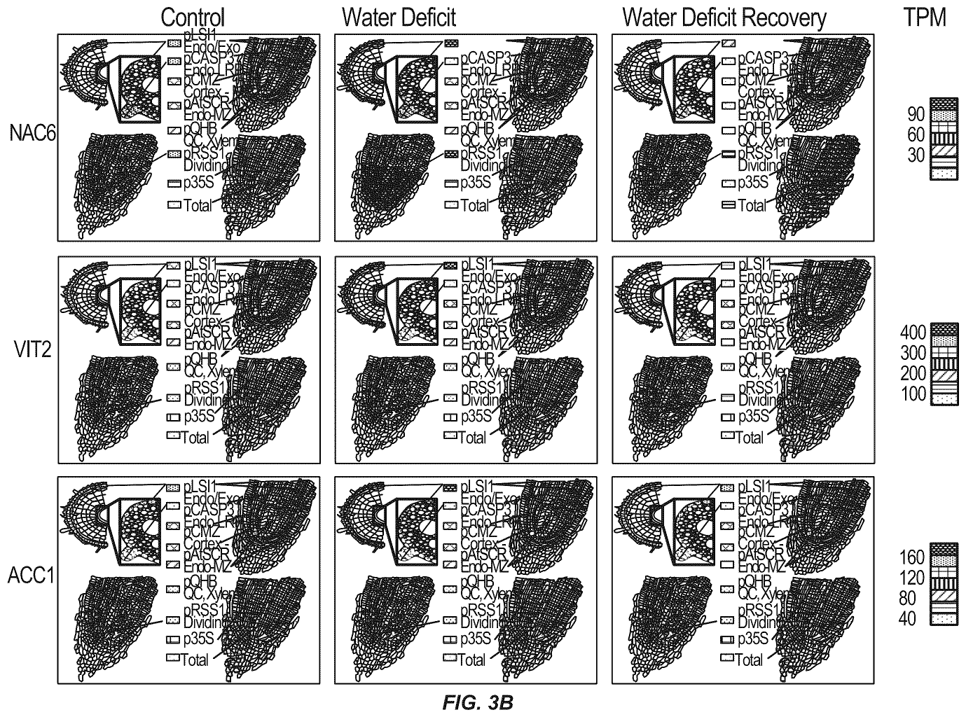

Next, we implemented a coexpression network approach using all of our translatome and polyA RNA data to define tightly coexpressed gene modules that represent activity of specific pathways in one or more cell populations. This identified small (<10 genes) to large (100+) modules. A 203 gene module in cells marked by pLSI1 (N5:MOD000731) likely is responsible for the water deficit-responsive suberin barrier, as it encompasses the biosynthetic pathway for suberin as currently understood, from monomer synthesis to transport and polymerization (FIG. 3A). The genes include the rice suberin transporter ABCG5 and enzymes whose functional roles have been validated for homologous proteins in other plant species such as Aliphatic Suberin Feruloyl Transferase (ASFT) and GDSL Esterase/Lipases (FIG. 3A). Implementing our pipeline that recognizes TFBSs in THSs proximal to genes on this coexpression module enabled construction of a water deficit-promoted suberin network of regulation dominated by MYB and NAC TFs (FIG. 3D). One of the MYB motifs (M07061) corresponds to the suberin-regulating A tMYB107.

Although it is well established that waterlogging increases exodermal suberization towards the tip of rice roots as a barrier to radial loss of oxygen, we did not observe upregulation of the suberin module in the waterlogging pLSI1 translatome. The upregulation of the suberin metabolic pathway transcripts and their translation may occur before the five-day time point when waterlogged plants were sampled. Alternatively, water deficit may require more suberin production to limit water loss due to increased root surface area compared to waterlogging, which fosters root growth near the soil surface for better aeration.

Suberin is a complex polymer which likely burdens the synthesizing cells with new metabolic demands, including an increased need for fatty acid biosynthesis by the plastid. Additionally, this barrier may require alterations in the apoplast and active transport pathways. To further investigate changes in the pLSI1-marked cells under water deficit, we looked at the different dominant patterns and gene modules from our analyses. Several patterns showed enrichment in these cells with pronounced downregulation under water deficit (data not shown). Staining across the whole root system significantly diminishes following water deficit. The Fe deficiency-response bHLH TFs IRO2, IRO3, and PRI1 are also enriched in pLSI1 translatomes and downregulated by water deficit as shown using a spatialHeatmap (FIG. 2B), suggesting that local Fe demand or uptake in LS//-marked cells is reduced during water deficit stress. IRO2 is exodermal but not endodermal expressed in rice, suggesting this module arises in the exodermis. Pattern 4 shows enrichment for bHLH and WRKY TFBSs (data not shown). Some members of these TF families show high transcript enrichment in pLSI1 and are water deficit downregulated and rewatering upregulated. These data illustrate conditional and reversible modulation of both TFs and target genes (FIG. 3B).

Metals including Fe are cofactors for reductive biochemical reactions, leading us to consider biochemical processes that may be altered to reduce the signature of Fe demand in the pLSI1 translatome under water deficit. Nitrogen (N) assimilation is an Fe-dependent process requiring the plastidal enzymes GOGAT, nitrite reductase (NiR), and an Fe-requiring ferredoxin cofactor; these genes are highly abundant in pLSI1 translatomes. Rice NiR (PSRI) was found in a coexpression module that includes the N assimilation-related gene ASNI (encoding asparagine synthetase) and a ferredoxin (FDVI) (N3:MOD001584). Transcripts in this pLSI1—enriched module peak during water deficit recovery. A translatome signature for N regulation is also enriched in the exodermis of tomato. These data support a model (FIG. 3E) wherein under control conditions the pLSI1-marked cells uptake Fe via IRT2, at least in part for incorporation into ferredoxin. Under water deficit, suberin biosynthesis is upregulated, requiring amplification of plastidal fatty acid biosynthesis. We hypothesize that this may be favored over N assimilation, leading to reduced Fe demand. This scenario is supported by the water deficit upregulation of ACETYL-COA CARBOXYLASE 1 (ACC1), the rate-limiting enzyme in fatty acid biosynthesis, and a vacuolar iron transporter (VIT2) (FIG. 3B), which may sequester excess iron in the vacuole under water deficit.

As observed for the metal ion module, the shift in N metabolism in the pLSI1 cell population appears to be reversible. Upon alleviation of water deficit stress, the peak in this N assimilation module indicates reprioritization of these processes, along with Fe uptake. These dynamics could reflect an inability of exodermal plastids to conduct both suberin biosynthesis and N assimilation simultaneously, or changes in cellular capacity to uptake Fe or N after additional suberization, or a combination of the two. Water deficit concomitantly limits N availability since ammonia and nitrates are water soluble, and there is a trade-off between N metabolism and water deficit tolerance, as decreasing N metabolism can promote water deficit tolerance and high N can promote root branching observed under water deficit. We hypothesize that the rice exodermis is critical for this interaction and is likely regulated by shifting activities of bHLH and WRKY TFs to MYB and NAC TFs under water deficit.

### Example 4. Hierarchical Regulation of Gene Expression in Cell Populations and Conditions

To infer which TFs might play key upstream regulatory roles versus those TFs that act downstream in transcriptional regulatory cascades, we profiled accessible chromatin in p35S, pQHB, and pCMZ-marked cells using INTACT:ATAC-seq, which in all cases yielded reads primarily in genic and promoter regions. Using 16 sets of paired ATAC-seq and TRAP-seq data across the three cell populations, we implemented a pipeline Taiji for gene regulatory network inference to predict TF that are important in each condition and cell population. Briefly, we mapped TF binding sites associated with 741 rice TF genes in the CisBP Database to the accessible chromatin regions that are located within 5 kb upstream or 1 kb downstream from a transcription start site. This generated a putative binding network for each of the 16 datasets that connected these TFs to target genes genome-wide. The network considers TF genes as both source and target nodes. Next, all genes were weighted based on relative transcript abundance in the corresponding TRAP-seq data. Finally, we ran the PageRank algorithm to re-weight this network, re-allocating weight to recognize key regulators. This identifies putative driver TFs: those that are more frequently regulating genes and other TFs expressed in the given cell population and condition. This identified 136 TFs with conditional and cell population relevance by clustering PageRank scores across all networks, with variable representation of TF families.

The Taiji analysis identifies distinct TF hierarchies in the p35S, pQHB, and pCMZ marked cells. The p35S ATAC-seq data highlight the ABRE-binding factors (bZIPs) as driver TFs with high PageRank specific to water deficit (cluster 8). This is consistent with their known activity and also establishes hierarchical relationships with other TFs, such as the drought-induced HOX22. Despite strong upregulation of HOX22 mRNA in the translatome, the hierarchy analysis predicts it is a secondary responder in the network: the bZIPs are predicted to activate HOX22 transcription, consistent with their role as direct first responders to ABA signaling. This water deficit-specific cluster includes several uncharacterized TFs. We also identified driver TFs present in the population of cells marked by pQHB, the quiescent center and developing xylem (clusters 4-6). Under control conditions, these include QHB itself, as well as conserved regulators of xylem development including four HD-ZIPIIIs, called HBs in rice (2 PHB/PHV-like and 2 REV-like, phylogenetically resolved in Kajala, K., Gouran, M., Shaar-Moshe, L., Mason, G. A., Rodriguez-Medina, J., Kawa, D., Pauluzzi, G., Reynoso, M., Canto-Pastor, A., Manzano, C., et al. (2021). Innovation, conservation, and repurposing of gene function in root cell type development. Cell), and three VND-like NACs, called SECONDARY WALL NACs (SWNs) in rice. The TF with the highest PageRank score in control conditions is the bHLH TMO5 LIKE 1 (TMO5L1) with orthology to AtTMO5, a key regulator of vascular development that has yet to be characterized in rice. HD-ZIPIIIs are predominant in the control condition-specific cluster 4, consistent with their downregulation under water deficit in Arabidopsis. The condition-stable cluster includes the TMO5-like and VND-like NACs. One TF of note in cluster 3, which reaches its peak PageRank score in the pCMZ domain under waterlogging, is RICE STARCH REGULATOR 1 (RSR1), which limits starch biosynthesis (Fu and Xue, 2010) and is predicted to bind the AP2 TFBS in the ground tissue network (data not shown).

This untargeted analysis illustrates TF hierarchies resolved by integration of ATAC-seq and TRAP-seq data from specific cell populations. It also identifies known responses (ABRE-binding factors) and conserved developmental regulators of xylem with high precision, validating the approach and suggesting the TFs we identify in other clusters likely have important condition and cell-population dependent roles. These relationships may control processes in specific cells that underlie conditional plasticity in physiology and development.

### Example 5. Demonstration of Function of Transcription Factors in Transactivation of Promoters of Genes of Suberin Biosynthesis

Methods

Transcription factor (TF) sequences were synthesized by Twist Biosciences and cloned downstream of the 35S promoter and an N-terminal mKOk fluorescent protein tag (Luginbuehl et al., 2020) or mTurquoise2 protein tag (Luginbuehl et al., 2020) and the NOS terminator using the MoClo system (Engler et al., 2014). Alternatively, the SV40 nuclear localization signal (CCCAAAAAGAAGAGGAAAGTTGGCGGGTGA (SEQ ID NO: 21)) was cloned using oligos synthesized by IDT. Promoter sequences of three rice suberin enzyme genes (CYP-LOC 0s04g47250 3.3 kb upstream of the transcription start site (TSS) (Nishiuchi et al., 2021); GPAT-LOC 0s02g02340 3 kb upstream of TSS (Nishiuchi et al., 2021); and ASFT-LOC 0s11g31090 2.5 kb upstream of the TSS;) were cloned by PCR from genomic DNA upstream of a GFP-GUS fusion protein with Gateway Cloning as described in (Reynoso et al., 2022).

Single TF transactivation evaluation was performed as follow: The vectors were transformed into Agrobacterium tumefaciens strain EHA105 and then co-infiltrated in equal densities into leaves of Nicotiana benthamiana. After 3 days, discs were cut and GFP and mKOk fluorescent signals were quantified on a plate reader. The log 2 of the ratio of GFP to mKOk plus one was compared across promoters and TF treatments to the NLS control using ANOVA and Tukey's HSD Test.

To evaluate interaction and independent effects, TFs constructs with distinct tags were co-infiltrated in N. benthamiana in combination with pASFT:GFP-GUS and compared to infiltration of pASFT:GFP-GUS alone and GFP fluorescence was measured as an output. ANOVA was used to test statistical significance of the impact of each TF independently and the interaction.

Results

TFs LOC 0s02g51799, LOC 0s06g02250, LOC 0s06g11780, LOC 0s05g50680 were all able to significantly activate at least one of these promoters (Table 2), supporting that these proteins can transactivate promoters of suberin biosynthesis genes. Both TFs

LOC 0s08g37970 and LOC 0s03g42630 independently transactivated the ASFT promoter (Table 3), supporting that they are independent transcriptional regulators of suberin biosynthesis gene promoters. There was no significance to their interaction (Table 3).

TABLE 2
Log2 Values of GFP/mKOk ratios for TF assays in Nicotiana benthamiana.

| | ASFT | | | CYP | | | GPAT | | |
|---|---|---|---|---|---|---|---|---|---|
| TF | Mean | SD | P-value | Mean | SD | P-value | Mean | SD | P-value |
| NLS | 0.14 | 0.016 | n.a. | 0.09 | 0.025 | n.a. | 0.08 | 0.004 | n.a. |
| LOC_Os02g51799 | 2.96 | 0.617 | *** | 1.30 | 0.408 | *** | 1.14 | 0.240 | *** |
| LOC_Os06g02250 | 2.92 | 0.600 | *** | 0.33 | 0.255 | * | 0.26 | 0.063 | *** |
| LOC_Os06g11780 | 3.20 | 0.743 | *** | 0.35 | 0.197 | ** | 0.70 | 0.054 | *** |
| LOC_Os05g50680 | 0.62 | 0.167 | *** | 0.09 | 0.022 | n.s. | 0.08 | 0.009 | n.s. |

P-values determined by ANOVA and Tukey's Highly Significant Differences Test, comparing the TF to NLS.
*** p < 0.001;
** p < 0.01;
* p < 0.05;
n.a.—not applicable;
n.s.—not significant.

TABLE 3
Measurement of the transactivation of the ASFT promoter
by LOC_Os03g42630 and LOC_Os08g3797

| | GFP | | | |
|---|---|---|---|---|
| | Mean | SD | | |
| No TF | 143 | 3693 | ANOVA | p-value |
| LOC_Os03g42630 | 2649 | 3568 | LOC_Os03g42630 | ** |
| LOC_Os08g37970 | 10007 | 9045 | LOC_Os08g37970 | *** |
| Both | 13262 | 19149 | Interaction | n.s. |

*** - p < 0.001;
** - p < 0.01;
* - p < 0.05;
n.a—not applicable;
n.s.—not significant.

### Example 6. CRISPR-Cas9 Mutagenesis and Phenotyping of Suberin

Rice plants with loss-of-function mutations were generated as described (Char et al., 2019). Segregation of transgene cassette was verified in T1 generation with PCR, and loss of function mutations were identified and confirmed with PCR and sanger sequencing of target genes. Rice plants were grown on ½ MS media with 1% sucrose for two days and then transferred to ½ MS media with 1% sucrose and 1 micromolar Abscisic Acid for two days. Suberin was stained with fluorol yellow 088 (0.01% in methanol) and visualized using a Typhoon biomolecular imager. Fluorescence intensity was quantified using Image.”

Mutants of LOC 0s02g51799 and LOC 0s11g31090 (ASFT), were also imaged at 28 days old with and without 8 days of water withholding. These plants show a strong reduction in drought-responsive suberization, indicative that this enzyme is required for rice root suberization (Table 4).

TABLE 4
Suberin measurements by fluorol yellow staining of mutants

| Genotype | Mean | Standard Deviation | P. value |
|---|---|---|---|
| Wild type | 8557.4 | 3091.9 | n.a. |
| LOC_Os02g51799 K.O. | 4705.2 | 2832.7 | ** |
| LOC_Os11g31090 K.O. | 1191.6 | 683.8 | *** |

P-values relative to wild type.
K.O.—Knockout;
*** - p < 0.001;
** - p < 0.01;
n.a—not applicable

### REFERENCES CITED IN EXAMPLE 5

Char, S. N., Li, R., and Yang, B. (2019). CRISPR/Cas9 for Mutagenesis in Rice. In Transgenic Plants: Methods and Protocols, S. Kumar, P. Barone, and M. Smith, eds (Springer New York: New York, NY), pp. 279-293.
Engler, C., Youles, M., Gruetzner, R., Ehnert, T.-M., Werner, S., Jones, J. D. G., Patron, N. J., and Marillonnet, S. (2014). A golden gate modular cloning toolbox for plants. ACS Synth. Biol. 3: 839-843.
Luginbuehl, L. H., El-Sharnouby, S., Wang, N., and Hibberd, J. M. (2020). Fluorescent reporters for functional analysis in rice leaves. Plant Direct 4: e00188.
Nishiuchi, S., Watanabe, K., Sato, S., Takahashi, H., and Nakazono, M. (2021). Expression analysis of genes for cytochrome P450 CYP86 and glycerol-3-phosphate acyltransferase related to suberin biosynthesis in rice roots under stagnant deoxygenated conditions. Plant Root 15: 19-35.
Reynoso, M. A. et al. (2022). Gene regulatory networks shape developmental plasticity of root cell types under water extremes in rice. Dev. Cell 57: 1177-1192.e6.

Exemplary sequences:

SEQ ID NO: 1: >LOC_Os01g67480 (OsBHLH109)
MNRMTAPHGGMPPPPMPAAGGLARYGSAPGSLLASIADSVIRGRGVGVVDQLHHHQHQHQ
LPPPPPPQQQQMVGRYFSAESSGLTSCESSCRTTTTTSTAAAADVGRHPLERAYGGSGEI
HVDASSAAVPLFRHSSSPAGLLSRLMADPHGNGMAATRGMGGYSGGGGDAGAMAHRRLSS
QWSFSRQDLPQISEMGGLIPDIGESIVTGGGGNSSSDGAGHGAQSSSFLSSRNFSMSSWD
DTNSIMFSPPSSSKKARVAAAAAGDHGDDMVSSFSNIDSQFGLSKQSSLEMAGMDDFLQL
QPDSVACRARAKRGCATHPRSIAERERRTRISKRLKKLQDLVPNMDKQTNTSDMLDIAVT
YIKELQGQVEKLKHDQANCTCSGKHDC*
SEQ ID NO: 2: >LOC_Os01g09080 (OsWRKY107)
MDVVVESPPVRDEKKVDVAAIGGAPPIVFESFAPSTQRDSTIIKKEEKMEAAKAEMGEVR
EENERLKTMLTRIVSDYKSLHTHFLDVVKVKEQTAAELSGDDDDDEPDDLVSLSLCTRPN
AAATRRKGHERTPSSGGGGDDGRLSLGLSCARGGVASDDDDDKQASRRALPPMPVLNLSS
DSSGDAAGAGAGEPTQPNKASRSSSGGGDGADDEVLQQQQAKKARVSVRVKCDTPTMNDG
CQWRKYGQKISKGNPCPRAYYRCTVAPNCPVRKQVQRCADDMSILITTYEGTHSHPLPPA
AAAMASTTSAAAAMLTSGSTNSTMHGSGGVHHHLPFASAVGGGGGVGLLGPTTISTATSC
PTVTLDLTAPHSLLHPSSASPYAAAAAGYESSRALPAAWSSGYLAYGGAAAAQPYYAKGV
APSPFGHHFGMMGMAAAAARPAPEQLFGGQTTSPYLQRAIGGGGVAPAAVTDTIAKAITS
DPSFQSVLAAAITSYMGRGGGAAAPNK*
SEQ ID NO: 3: >LOC_Os03g42630 (ONAC6)
MEEGLPPGFRFHPTDEELVTYYLARKVSDFGFATRAIADVDLNKCEPWDLPSKASMGEKE
WYFFSMRDRKYPTGIRTNRATDSGYWKTTGKDKEIFHGGALAGMKKTLVFYRGRAPKGAK
TSWVMHEYRLQSKFPYKPAKDEWVVCRVFKKLQCHLAKPRPPHDDVDGDGASPPEMVDAS
SLGELGELDVSSILLGGFAPPSGELCHGGGGGDGFGAHRLHVGAYMSWLQAAAAANQGMF
QWPAATQAGLVGGTVFAAAHKAAGTMPFGGGCSQQQARDVGVSLANVGGGDALFGGAPLA
KVDMECGEQAPQLDMDDSTWRAF*
SEQ ID NO: 4: >LOC_Os01g32920 (ZOS1-08)
MAGVVQTVQCDQPSKQQLTEAAEAAAAAAAAASLRIFGYEVGGGGGGGGVAAAAGARREA
ADVVVGAGRRFECQYCCREFANSQALGGHQNAHKKERQQLKRARLQLAVAAAGTPAGMGF
AAAQAPAPPPGHVIAVGHHAGSVASYALPRWVYLAAHHLQPPAVGLPFHATVQAAAGACH
GGGDVETRLLSVAAAGRGSSTAGSSAHSYEVCAPAAPADDSDEEASAMGLDLHLSLAPAS
SA*
SEQ ID NO: 5: >LOC_Os05g45230 (OsWRKY58)
MDGLEAAAGDQQHGRLLIPQLPAAYLASSSMAALSPAGDDWAASLILPDGGSAAAGVGED
DLGGGVMAAAAAESSCGGSSTVTSSGVTEAAAAAATTTRRGRGNGKKAGGGGRTPRFAFH
TRSENDILDDGYRWRKYGQKAVKNSDFPSDDELLLFSDVDNTQTATENLRFIPLGRVYIT
G*
SEQ ID NO: 6: >LOC_Os05g50680 (OsWRKY83)
MSPWRPGTPPRRAKHRLPPPPTTPISRGPRSMAVSGVGAGAARGLLFTSPSTAPAPPAPA
LSAPRSCHLRCAGQGREGCHQEQGSRWEAAVAVVQLLVLHRHRRRAPAVQQQRRGGRRGG
GDEVVDALLLPQHLLRLHLRFLQQHRRRVKAPPQEPTSPRASPRAAERRQCRRREAARRQ
QGRREESRRQARRRRRRRGRGGQHGGGEAVAQPVRRLPELDGGDGGGAAHLWRRRHGRPP
DVVPVAQLKAPPPGHPRRLRGRLGSRLRHALIDRSIDRSPSL*
SEQ ID NO: 7: >LOC_Os02g51799 (n/a)
MGRSPCCDENGLKKGPWTTEEDEKLMEYIQKNGHGSWRALPKLAGLNRCGKSCRLRWTNY
LRPDIKRGKFTSAEKDTILQLHAVLGNKWSAIAKHLPGRTDNEIKNYWNTHLKKDLIQKG
IDPTTHRPRTDFFAALPQLIALANLRQLVGQLPWDDPAAAAAGLQQAEAAQAAKFGQYLH
LQALLQPPPPSAATSPRSGCIGGAAAAMPGGDMEQMGAGLLSPPAMSSAALSPMSTSSLS
PLPSPMSSTALSPLPPSSFFGNGGQGSFLAGQLPNIQMHGSSLFDQSAAIINDANHNQDY
AASTGEREIINGATKTTTTTLLPEGSLPPLADYPTISNLGDVYSTPSCDGNITEFPLLPD
ALFDELMRDYVSPSSSF*
SEQ ID NO: 8: >LOC_Os06g11780 (n/a)
MGRPPCCDENGLKKGPWTPEEDEKLMSYIQKHGHASWRVLPELAGLNRCGKSCRLRWTNY
LKPDIKRGNFSREEEQTILQLHSILGNKWSAIAKHLPGRTDNEIKNFWNTHLRKKLIKMG
IDPMTHCPRTDFFQSLPQLIALANLRQIIERQPWDGHIEGLQTVAVQAAKLEYMQSLLHS
AVSIVTSPTTTTTTSSLNTFTTELEQTNHLCPPQVPSSSVPELAVQVPHSQMPSTSFDQE
IGKTNLFSNNIVNGNEWCSMEADNSSQKSLLVPENSIPPLIDMPVQNFCNTISTPNCDGG
NSIPLPSWSEILLDEELMGEFA*
SEQ ID NO: 9: >LOC_Os08g37970 (n/a)
MGRSPCCDESGLKKGPWTPEEDEKLLHYIQKNGHGSWRTLPRLAGLNRCGKSCRLRWTNY
LRPDIKRGKFSQEEEQTILHLHSILGNKWSAIATHLPGRTDNEIKNFWNTHLKKRLIQMG
FDPMTHRPRTDFFAALPQLIALATLRDQLAATACGGGGGGDTAAAAAAQIMQGAGGGGGA
DVAIQAAKMQYLQCLLQSAASTIASAGSGGGGGGAMSAHDADAALAAAAAAAATCSSSPA
GSSVPITPTAVGHSGGGQTQTTPCAFSEAPVITSDDVEANLRLLGCGAGGADAFTCHGGS
LPPLADLSDVTTTTMTNPLDWCSATASASSCGGGGGASSPFPWPEFFHDDPFITDFL*
SEQ ID NO: 10: >LOC_Os06g02250 (n/a)
MGRSPCCEQDADVKKGPWTPEEDKLLVEYIGKNGHGSWRRLPKLAGLNRCGKSCRLRWTN
YLRPDIKRGGFSDDEERLIIHLHATLGNKWSSIATKLKGRTDNEIKNYWNTHLRKKLLSQ
GIDPVTHRPRTDLLAGLPNLLAAANLGGAAHQLPLDLNAIKLQADAAKFQILQGLLRVLA
STTAPPPTAAVLPGTDLMTSILGATLAANSAGILGQQQQLAGVDLSRLGQYNGNYDNLPP
LTNDSCTQQTQPAMSSMSPDSLLNRISSGISGDMLGSPELCHGGDGLSSPELGQGGPSAS
NMTTSPMAPPPPMVAADDHQCNTNTPSGGGDGMSCEQTPASSTFDGLNLDDIDMEGCWAM
TDILLAE*
SEQ ID NO: 11: >LOC_Os11g31090 (OsASFT)
MVVEVRENGAVVEVADGGKAAAAAKLTVKRGEPELVAPAEATPTGEKYYLSNLDQNIAVI
VQTVYCYKPPAASGGDNGDAVAVLRDALAKVLVHYHPLAGRLTISAEMKLAVELTGEGAV
FVAADAGYDLADVGDLTKPDPAALGHLVYSIPGAKNILEMPPMTAQVTRFKCGGFALGLA
MNHCMFDGLGAMEFVNSWAETARGAAELTVPPFLDRTLLRARDPPVISFEHHEFEEIPDV
SDTAALYADQDLLYRSFCFDPDRLERVRALALAGAGAENGDDLVGGRCTTFEALSGLVWR
ARTRALGLAPEQRTKLLFAVDGRRRFEPPLPRGYFGNGIVLTNAVATAGELLSSPPSRAA
GLVQAAVRMVTDGYMRSAVDYFEATRARPSLASTLLITTWSRLAFHGADFGWGAPAMSGP
VTLPEKEVILFLAHGEERKSINVLLGLPASAMDAFQELMDEI*
SEQ ID NO: 12: >LOC_Os05g38350 (n/a)
MVTMSASAEAKSRRERRSVVAELEGALLRDAATFPYFMLVAFEASGLPRFAALLALWPLL
WALERALGRGDLALRAAAFVATAGVPRAEVEAVARAVLPKFMADDVDPAAWAAFGSCGGR
RVVVTRMPRVMVERFAREHLGAHEVVGCDLEYSRLRRSTGFVRGGGGGERAVAERVRALF
ADGDRPDVGIARSESATRSFLPFCKKQLRPPFCEDDGDDVAAAGEQHKCPPFRPVIFHDG
RLVCRPTPLMSLVILLWLPLGALVAFVRIAVGISVPIQIIPRIAPYFGGAVVVHGAPPPA
AASGPASGVLFVCTHRTLMDPVVLATVLGRRVAAVTYSISRLSEVLSPIPTVRLTRDRGV
DAAQMRAELSRGDVAVCPEGTTCREPFLLRFSALFAELSDRIVPVAMNYRVGLFHPTTAR
GWKAMDPIFFFMNPRPVYEVTFLNQLPAEETCAAGKSAVDVANYVQRILAAKLGFECTTF
TRKDKYRVLAGNDGIVNVKPPAAADPAPWQRRLKEVLGFLLH*
SEQ ID NO: 13: >LOC_Os01g63580 (n/a)
MVSRRFKPVEECSSDGRSEQTVAADFDGTLVRSRSAFPYYLLVALEAGSVLRAVVLLLSV
PFVYVTYIFFSESLAISTLVYISVAGLKVRNIEMVARSVLPKFYAEDVHPESWRVENSFG
KRYIITASPRIMVEHFAKTFLGADKVVGTELEVGKNGKATGFMVKPGVLVGDHKRQAVVK
ELRDAVPDVGLGDRETDFDFMSICKEAYLVTSRKYSAVPKNQLLSPLILHDGRLVQRPTP
LVALVTFLWMPFGFALALLRVYVNLPLPERIVFYTYKLMGIRLIVKGNPPPPPKKGHPGV
LFVCNHRTVLDPVEVAVALRRKVSCVTYSISKFSELISPIKAVALSREREKDAENIRRLL
EEGDLVICPEGTTCREPFLLRFSALFAELTDRIVPVAINTKESMFHGSTVRGFKLMDPYF
FFMNPRPTYEITFLNQLPKELTCSGGKSPIEVANYIQKTLSGQLGFECTAITRKEKYSIL
AGTDGRVPSKNKEKEKN*
SEQ ID NO: 14: LOC_Os01g46750 (OsLACS5)
MEMQHVVEVEQGRAAADGRPSVGPAYRSAFARGGFPPPVAGLDCCYDIFRMAVEKFPDNR
MLGHREIVDGKAGAYVWKTYKEVFDLSTKIGNSIRSRGLAKGSRCGIYGANCPEWIITME
ACNAHGIYCVPLYDTLGAGAVEFILCHAEVEIAFTEEKKIEQIFKTFPKSAEFLKTIVSF
GKVTQEQKENASKYGLEIYSWDEFLSLADQEFDLPVKAKTDICTIMYTSGTTGDPKGVLI
SNASIICLVAGVDRLLNCVNEQLEQTDVYMSYLPLAHIFDRVVEELFMFHGASIGFWRGD
VKLLVEDIGTLKPTILCAVPRVLDRIFSGLQAKIASGGFIKSTLFNLAYKFKQFRMMKGA
KHNEAAAICDKVVFSKVKEGLGGNVRVILSGAAPLATHVEEYLRVVTCAHVLQGYGLTET
CAGSFVSLPNQMCMIGTVGPPVPNIDVCLESVPEMNYDALATRPRGEICIRGETVFSGYY
KREDLTKDVLIDGWFHTGDIGEWQPDGSMKIIDRKKNIFKLSQGEYVAVENLENIYGLVS
AIDSIWVYGNSFESFLVAVVNPNKEALESWAAANGISGDLEALCENPKAKEYILGELSKV
GKEKKLKGFEFIKAVHLEPVPFDMDRDLITPTYKKKRPQLLKYYQGTIDNMYRSAK*
SEQ ID NO: 15: >LOC_Os02g44654 (n/a)
MEVGTWAVVVAVAAAYMTWFWRMSRGLSGPRVWPVVGSLPGLVQHAENMHEWIAANLRRA
GGTYQTCIFAVPGVARRGGLVTVTCDPRNLEHVLKSRFDNYPKGPFWHAVFRDLLGDGIF
NSDGETWVAQRKTAALEFTTRTLRTAMSRWVSRSIHHRLLPILDDAAAGKAHVDLQDLLL
RLTFDNICGLAFGKDPETLAKGLPENAFASAFDRATEATLNRFIFPEYLWRCKKWLGLGM
ETTLASSVAHVDQYLAAVIKARKLELAGNGKCDTVAMHDDLLSRFMRKGSYSDESLQHVA
LNFILAGRDTSSVALSWFFWLVSTHPAVERKVVHELCAVLAASRGAHDPALWLAAPFTFE
ELDSLVYLKAALSETLRLYPSVPEDSKHVVADDYLPDGTFVPAGSSVTYSIYSAGRMKTV
WGEDCLEFRPERWLSADGSKFEPHDSYKFVAFNAGPRICLGKDLAYLQMKNIAGSVLLRH
RLAVAQGHRVEQKMSLTLFMKNGLRMEVRPRDLAPVADELRGADVRATAPCA*
SEQ ID NO: 16: >LOC_Os04g47250 (n/a)
MEAGTWAVVVAAVAAYMAWFWRMSRGLSGPRVWPVVGSLPGLVRHAEDMHEWIAANLRRT
RGTYQTCIFAVPGLARRGGLVTVTCDPRNLEHVLKSRFDNYPKGPFWHGVFGDLLGDGIF
NSDGETWVAQRKTAALEFTTRTLRTAMSRWVSRSIHSRLLPILSDAAAAGGGGGGATVDL
QDLLLRLTFDNICGLAFGKDPETLARGLPENDFASAFDRATEATLNRFIFPECVWRFKKW
MGLGMETTLARSVQHVDRYLSAVIKARKLELAAGNGKGDASSATPHDDLLSRFMRKGTYS
DESLQHVALNFILAGRDTSSVALSWFFWLVSTHPAVERKIVRELCTVLAASRGADDPALW
LAAPLNFEELDQLVYLKAALSETLRLYPSVPEDSKHVVADDVLPDGTFVPAGSSVTYSIY
SAGRMKTVWGDDCLEFRPERWLSADGTKFEPHDSFRFVAFNAGPRICLGKDLAYLQMRNI
AGSVLLRHRLAVAPGHRVEQKMSLTLFMKHGLRMEVRPRDLAPIVDELRGAGEYAAAARA
TAACA*
SEQ ID NO: 17: >LOC_Os08g44360 (n/a)
MMASDRLDAERIIEYFKGKSILITGATGFLGKILVEKILRVQPDVKKIYLLVRAIDQASA
NQRVQSEVTETELFSVVKEKHGKGFSRFIEEKVVALAGDIIYDDLGLDPPLLQHLADNLD
VIVNGAATTNFYGRYDVSLDVNVLGVKHLCQLAKKCRGLKVFLHVSTAYAGGEQEGLIQE
RAFEEGWALREGMHLDVDAELRLVADVRREVEDDDDKARRKAMKELGLARARHFGWSNTY
VFTKAMGEMVLSRMLLQSSSPAPAITGAGGVVVVRPSIITSIQRDPVPGWMQGTRTIDTL
IIGYAKQNLSCFLGDLDLVMDVIPGDMVVNAMMAAAVAHSGEAGQERPAVYHVSSSLRNP
AAYSVLYEAGRRHFTEKPRVGKRGEVIPTKEMHFFKTIASFQVYMLVKYRLPLEILHLVN
LLLCGLFSRLYSNLARQYRYVMHLVDVYGPFAFFKGCFDDINLERLRQRMGKSRNPQDDE
MFNFDPKTIDWEDYFYRIHIPGVLKYILK*
SEQ ID NO: 18: >LOC_Os09g39410 (n/a)
MVGTMDEARIIEYFKNKSILITGSTGFLGKILVEKILRVQPDVRKIYLPVRAVDAATAKQ
RMQTEVIGKELFGLLKEQHGKGFQSFIDEKVVPLAADMMHQNLGLEESTLQELAKDLNII
VNGAATTNFYERYDVALDVNVMGVKYLCQLAKKCANLEVFLHVSTAYVCGERSGVVQERA
LREGETLREGTYLDIETELRLVGEQRQQLEDAGDAKAERKAMKDLGLARARHFGWPNTYV
FTKAMGEMMLQEQLVAGAGRRHGIPVVIARPSIITSVHRDPLPGWIEGTRTIDAIIIGYA
KQSLSCFLADLDLIMDVVPGDLVVNAMMAAMVAHSRGSSSEMAVYHVTSSMRHPAAYAVL
YRTGWRYFLENPRVGKDGVAVRTRPVYFFRTIASFRAFMAVAYALPLQLLRLLSLLCFGL
LFARRYADLSRKYSFVMQLVDLYGPFALFKACFDDLNMEKLRLSMATPPSSAAAALFNLD
PKNIDWDDYFYRIHIPGVMKYVLNK*
SEQ ID NO: 19: >LOC_Os11g37900 (n/a)
MKLLYHHAISNAPYLMLAAAAAAVALRASRLAPADDLATARELLATNLPLAVALLAAAVV
LATAYLMRRPRPVYLLDFACYKPGPEHVVTRETFMAQSAAAGAFTGDSLAFQRKILERSG
LGQGTYFPAAVLNSPPNPCMAEARREAEQVMFGAIDAVLAKTGVRARDIGVVVVNCSLFN
PTPSLSAMIVNHYKLRGNVATYNLGGMGCSAGLISIDLAKQLLQVHRNSYALVVSMENIT
LNWYWGNNRSMLVSNCLFRMGGAAILLSNRGGDRRRAKYQLLHTVRTHSGAADRAYRCVF
QEEDDAAGVGVALSKDLMAVAGEALRTNITTLGPLVLPMSEQILFLASLVARRVFGLAGV
RPYIPDFKMAFDHFCIHAGGRAVLDTIEKNLGLGAWHMEPSRMTLYRWGNTSSSSLWYEL
AYAEAKGRVRRGQTAWQIAFGSGFKCNSAVWRALRTVEPDADERNPWAGEIDSFPVEVPK
VEAVATATADAASS*
SEQ ID NO: 20: >LOC_Os04g50680 (n/a)
MGRSPCCCHDAGVKKGPWTEEEDRALVEHIKKQGGHVGSWRGLPRAAGLNRCGKSCRLRWTN
YLRPDIRRGNFSDDEERLIIRLHAALGNKWSTIATHLDGRTDNEIKNYWNTHIKKKLLRMGI
DPVTHQRLPPDLLADGGGLGAASPLLSPPGPAAAAALQPLLSAVASLGSLDTALRQFQLLQH
LLNSITSSSSDVAATAGLMATNLAATNTMVNSSSNVASFQEQMNALAHANYQPGYLRDVVPS
FPGQDMAPQLNSTSSTPSTAPVLRSSAEPADQCCNDAALVPETYPREVAASVDHWKVQDFPS
LEPLELPNLSTLESDLDPFWKEILESSFRS*

What is claimed is:

1. A plant having increased suberin, wherein the plant ectopically expresses or overexpresses one or more polypeptides comprising an amino acid sequence having at least 90% identity to an amino acid sequence of any one of SEQ ID NOS:3-20, wherein the plant has increased suberin compared to a control plant not ectopically expressing or overexpressing the one or more polypeptides.

2. The plant of claim 1, wherein the amino acid sequence comprises any one of SEQ ID NOS:3-20.

3. The plant of claim 1, wherein the amino acid sequence has at least 90% identity to any one of SEQ ID NO:3, 6, 7, 8, 9, 10 or 11.

4. The plant of claim 1, wherein the sequence comprises the amino acid sequence of any one of SEQ ID NO:3, 6, 7, 8, 9, 10 or 11.

5. The plant of claim 1, wherein the plant is a graminaceous plant.

6. The plant of claim 1, wherein the plant is a rice plant.

7. The plant of claim 1, wherein the plant comprises an expression cassette comprising a promoter operably linked to a polynucleotide encoding one or more polypeptides having at least 90% identity to any one of SEQ ID NO:3-20.

8. The plant of claim 7, wherein the polypeptide comprises any one of SEQ ID NOS:3-20.

9. The plant of claim 7, wherein the polypeptide comprises a sequence having at least 90% identity to any one of SEQ ID NO:3, 6, 7, 8, 9, 10 or 11.

10. The plant of claim 7, wherein the polypeptide comprises a sequence of any one of SEQ ID NO:3, 6, 7, 8, 9, 10 or 11.

11. The plant of claim 7, wherein the promoter is inducible or a growth-stage or tissue- or cell-specific promoter.

12. A part of the plant of claim 1.

13. A method of making suberin, the method comprising:
providing the plant or a part of the plant of claim 1; and
extracting suberin from the plant or the part of the plant.

14. A method of cultivating plants that are tolerant to high salinity, drought or flooding conditions, the method comprising cultivating the plant of claim 1 under high salinity, drought, or flooding conditions.

15. A plant having decreased suberin, wherein the plant (a) is mutated or engineered to reduce or knockout expression of, or (b) expresses an siRNA or antisense polynucleotide to reduce expression of, or (c) expresses a modified endonuclease such as Cas9 targeted to reduce expression of one or more polypeptides comprising a sequence having at least 90% identity to an amino acid sequence any one of SEQ ID NOS:3-20, wherein the plant has decreased suberin compared to a control plant that expresses the one or more polypeptides.

16. The plant of claim 15, wherein the one or more polypeptides comprise a sequence of any one of SEQ ID NO:3-20.

17. The plant of claim 15, wherein the amino acid sequence has at least 90% identity to any one of SEQ ID NO:3, 6, 7, 8, 9, 10 or 11.

18. The plant of claim 15, wherein the one or more polypeptides comprises the amino acid sequence of any one of SEQ ID NO:3, 6, 7, 8, 9, 10 or 11.

19. The plant of claim 15, wherein the plant is a Graminaceous plant.

20. The plant of claim 15, wherein the plant is a rice plant.


SEQUENCE LISTING (21 sequences)

SEQ ID NO: 1 - protein, 387 aa, Oryza sativa
MNRMTAPHGGMPPPPMPAAGGLARYGSAPGSLLASIADSVIRGRGVGVVDQLHHHQHQHQLPPPPPPQQQQMVGRYFSAESSGLTSCESSCRTTTTTSTAAAADVGRHPLERAYGGSGEIHVDASSAAVPLFRHSSSPAGLLSRLMADPHGNGMAATRGMGGYSGGGGDAGAMAHRRLSSQWSFSRQDLPQISEMGGLIPDIGESIVTGGGGNSSSDGAGHGAQSSSFLSSRNFSMSSWDDTNSIMFSPPSSSKKARVAAAAAGDHGDDMVSSFSNIDSQFGLSKQSSLEMAGMDDFLQLQPDSVACRARAKRGCATHPRSIAERERRTRISKRLKKLQDLVPNMDKQTNTSDMLDIAVTYIKELQGQVEKLKHDQANCTCSGKHDC

SEQ ID NO: 2 - protein, 507 aa, Oryza sativa
MDVVVESPPVRDEKKVDVAAIGGAPPIVFESFAPSTQRDSTIIKKEEKMEAAKAEMGEVREENERLKTMLTRIVSDYKSLHTHFLDVVKVKEQTAAELSGDDDDDEPDDLVSLSLCTRPNAAATRRKGHERTPSSGGGGDDGRLSLGLSCARGGVASDDDDDKQASRRALPPMPVLNLSSDSSGDAAGAGAGEPTQPNKASRSSSGGGDGADDEVLQQQQAKKARVSVRVKCDTPTMNDGCQWRKYGQKISKGNPCPRAYYRCTVAPNCPVRKQVQRCADDMSILITTYEGTHSHPLPPAAAAMASTTSAAAAMLTSGSTNSTMHGSGGVHHHLPFASAVGGGGGVGLLGPTTISTATSCPTVTLDLTAPHSLLHPSSASPYAAAAAGYESSRALPAAWSSGYLAYGGAAAAQPYYAKGVAPSPFGHHFGMMGMAAAAARPAPEQLFGGQTTSPYLQRAIGGGGVAPAAVTDTIAKAITSDPSFQSVLAAAITSYMGRGGGAAAPNK

SEQ ID NO: 3 - protein, 323 aa, Oryza sativa
MEEGLPPGFRFHPTDEELVTYYLARKVSDFGFATRAIADVDLNKCEPWDLPSKASMGEKEWYFFSMRDRKYPTGIRTNRATDSGYWKTTGKDKEIFHGGALAGMKKTLVFYRGRAPKGAKTSWVMHEYRLQSKFPYKPAKDEWVVCRVFKKLQCHLAKPRPPHDDVDGDGASPPEMVDASSLGELGELDVSSILLGGFAPPSGELCHGGGGGDGFGAHRLHVGAYMSWLQAAAAANQGMFQWPAATQAGLVGGTVFAAAHKAAGTMPFGGGCSQQQARDVGVSLANVGGGDALFGGAPLAKVDMECGEQAPQLDMDDSTWRAF

SEQ ID NO: 4 - protein, 242 aa, Oryza sativa
MAGVVQTVQCDQPSKQQLTEAAEAAAAAAAAASLRIFGYEVGGGGGGGGVAAAAGARREAADVVVGAGRRFECQYCCREFANSQALGGHQNAHKKERQQLKRARLQLAVAAAGTPAGMGFAAAQAPAPPPGHVIAVGHHAGSVASYALPRWVYLAAHHLQPPAVGLPFHATVQAAAGACHGGGDVETRLLSVAAAGRGSSTAGSSAHSYEVCAPAAPADDSDEEASAMGLDLHLSLAPASSA

SEQ ID NO: 5 - protein, 181 aa, Oryza sativa
MDGLEAAAGDQQHGRLLIPQLPAAYLASSSMAALSPAGDDWAASLILPDGGSAAAGVGEDDLGGGVMAAAAAESSCGGSSTVTSSGVTEAAAAAATTTRRGRGNGKKAGGGGRTPRFAFHTRSENDILDDGYRWRKYGQKAVKNSDFPSDDELLLFSDVDNTQTATENLRFIPLGRVYITG

SEQ ID NO: 6 - protein, 282 aa, Oryza sativa
MSPWRPGTPPRRAKHRLPPPPTTPISRGPRSMAVSGVGAGAARGLLFTSPSTAPAPPAPALSAPRSCHLRCAGQGREGCHQEQGSRWEAAVAVVQLLVLHRHRRRAPAVQQQRRGGRRGGGDEVVDALLLPQHLLRLHLRFLQQHRRRVKAPPQEPTSPRASPRAAERRQCRRREAARRQQGRREESRRQARRRRRRRGRGGQHGGGEAVAQPVRRLPELDGGDGGGAAHLWRRRHGRPPDVVPVAQLKAPPPGHPRRLRGRLGSRLRHALIDRSIDRSPSL

SEQ ID NO: 7 - protein, 377 aa, Oryza sativa
MGRSPCCDENGLKKGPWTTEEDEKLMEYIQKNGHGSWRALPKLAGLNRCGKSCRLRWTNYLRPDIKRGKFTSAEKDTILQLHAVLGNKWSAIAKHLPGRTDNEIKNYWNTHLKKDLIQKGIDPTTHRPRTDFFAALPQLIALANLRQLVGQLPWDDPAAAAAGLQQAEAAQAAKFGQYLHLQALLQPPPPSAATSPRSGCIGGAAAAMPGGDMEQMGAGLLSPPAMSSAALSPMSTSSLSPLPSPMSSTALSPLPPSSFFGNGGQGSFLAGQLPNIQMHGSSLFDQSAAIINDANHNQDYAASTGEREIINGATKTTTTTLLPEGSLPPLADYPTISNLGDVYSTPSCDGNITEFPLLPDALFDELMRDYVSPSSSF

SEQ ID NO: 8 - protein, 322 aa, Oryza sativa
MGRPPCCDENGLKKGPWTPEEDEKLMSYIQKHGHASWRVLPELAGLNRCGKSCRLRWTNYLKPDIKRGNFSREEEQTILQLHSILGNKWSAIAKHLPGRTDNEIKNFWNTHLRKKLIKMGIDPMTHCPRTDFFQSLPQLIALANLRQIIERQPWDGHIEGLQTVAVQAAKLEYMQSLLHSAVSIVTSPTTTTTTSSLNTFTTELEQTNHLCPPQVPSSSVPELAVQVPHSQMPSTSFDQEIGKTNLFSNNIVNGNEWCSMEADNSSQKSLLVPENSIPPLIDMPVQNFCNTISTPNCDGGNSIPLPSWSEILLDEELMGEFA

SEQ ID NO: 9 - protein, 357 aa, Oryza sativa
MGRSPCCDESGLKKGPWTPEEDEKLLHYIQKNGHGSWRTLPRLAGLNRCGKSCRLRWTNYLRPDIKRGKFSQEEEQTILHLHSILGNKWSAIATHLPGRTDNEIKNFWNTHLKKRLIQMGFDPMTHRPRTDFFAALPQLIALATLRDQLAATACGGGGGGDTAAAAAAQIMQGAGGGGGADVAIQAAKMQYLQCLLQSAASTIASAGSGGGGGGAMSAHDADAALAAAAAAAATCSSSPAGSSVPITPTAVGHSGGGQTQTTPCAFSEAPVITSDDVEANLRLLGCGAGGADAFTCHGGSLPPLADLSDVTTTTMTNPLDWCSATASASSCGGGGGASSPFPWPEFFHDDPFITDFL

SEQ ID NO: 10 - protein, 367 aa, Oryza sativa
MGRSPCCEQDADVKKGPWTPEEDKLLVEYIGKNGHGSWRRLPKLAGLNRCGKSCRLRWTNYLRPDIKRGGFSDDEERLIIHLHATLGNKWSSIATKLKGRTDNEIKNYWNTHLRKKLLSQGIDPVTHRPRTDLLAGLPNLLAAANLGGAAHQLPLDLNAIKLQADAAKFQILQGLLRVLASTTAPPPTAAVLPGTDLMTSILGATLAANSAGILGQQQQLAGVDLSRLGQYNGNYDNLPPLTNDSCTQQTQPAMSSMSPDSLLNRISSGISGDMLGSPELCHGGDGLSSPELGQGGPSASNMTTSPMAPPPPMVAADDHQCNTNTPSGGGDGMSCEQTPASSTFDGLNLDDIDMEGCWAMTDILLAE

SEQ ID NO: 11 - protein, 462 aa, Oryza sativa
MVVEVRENGAVVEVADGGKAAAAAKLTVKRGEPELVAPAEATPTGEKYYLSNLDQNIAVIVQTVYCYKPPAASGGDNGDAVAVLRDALAKVLVHYHPLAGRLTISAEMKLAVELTGEGAVFVAADAGYDLADVGDLTKPDPAALGHLVYSIPGAKNILEMPPMTAQVTRFKCGGFALGLAMNHCMFDGLGAMEFVNSWAETARGAAELTVPPFLDRTLLRARDPPVISFEHHEFEEIPDVSDTAALYADQDLLYRSFCFDPDRLERVRALALAGAGAENGDDLVGGRCTTFEALSGLVWRARTRALGLAPEQRTKLLFAVDGRRRFEPPLPRGYFGNGIVLTNAVATAGELLSSPPSRAAGLVQAAVRMVTDGYMRSAVDYFEATRARPSLASTLLITTWSRLAFHGADFGWGAPAMSGPVTLPEKEVILFLAHGEERKSINVLLGLPASAMDAFQELMDEI

SEQ ID NO: 12 - protein, 522 aa, Oryza sativa
MVTMSASAEAKSRRERRSVVAELEGALLRDAATFPYFMLVAFEASGLPRFAALLALWPLLWALERALGRGDLALRAAAFVATAGVPRAEVEAVARAVLPKFMADDVDPAAWAAFGSCGGRRVVVTRMPRVMVERFAREHLGAHEVVGCDLEYSRLRRSTGFVRGGGGGERAVAERVRALFADGDRPDVGIARSESATRSFLPFCKKQLRPPFCEDDGDDVAAAGEQHKCPPFRPVIFHDGRLVCRPTPLMSLVILLWLPLGALVAFVRIAVGISVPIQIIPRIAPYFGGAVVVHGAPPPAAASGPASGVLFVCTHRTLMDPVVLATVLGRRVAAVTYSISRLSEVLSPIPTVRLTRDRGVDAAQMRAELSRGDVAVCPEGTTCREPFLLRFSALFAELSDRIVPVAMNYRVGLFHPTTARGWKAMDPIFFFMNPRPVYEVTFLNQLPAEETCAAGKSAVDVANYVQRILAAKLGFECTTFTRKDKYRVLAGNDGIVNVKPPAAADPAPWQRRLKEVLGFLLH

SEQ ID NO: 13 - protein, 497 aa, Oryza sativa
MVSRRFKPVEECSSDGRSEQTVAADFDGTLVRSRSAFPYYLLVALEAGSVLRAVVLLLSVPFVYVTYIFFSESLAISTLVYISVAGLKVRNIEMVARSVLPKFYAEDVHPESWRVFNSFGKRYIITASPRIMVEHFAKTFLGADKVVGTELEVGKNGKATGFMVKPGVLVGDHKRQAVVKELRDAVPDVGLGDRETDFDFMSICKEAYLVTSRKYSAVPKNQLLSPLILHDGRLVQRPTPLVALVTFLWMPFGFALALLRVYVNLPLPERIVFYTYKLMGIRLIVKGNPPPPPKKGHPGVLFVCNHRTVLDPVEVAVALRRKVSCVTYSISKFSELISPIKAVALSREREKDAENIRRLLEEGDLVICPEGTTCREPFLLRFSALFAELTDRIVPVAINTKESMFHGSTVRGFKLMDPYFFFMNPRPTYEITFLNQLPKELTCSGGKSPIEVANYIQKTLSGQLGFECTAITRKEKYSILAGTDGRVPSKNKEKEKN

SEQ ID NO: 14 - protein, 656 aa, Oryza sativa
MEMQHVVEVEQGRAAADGRPSVGPAYRSAFARGGFPPPVAGLDCCYDIFRMAVEKFPDNRMLGHREIVDGKAGAYVWKTYKEVFDLSTKIGNSIRSRGLAKGSRCGIYGANCPEWIITMEACNAHGIYCVPLYDTLGAGAVEFILCHAEVEIAFTEEKKIEQIFKTFPKSAEFLKTIVSFGKVTQEQKENASKYGLEIYSWDEFLSLADQEFDLPVKAKTDICTIMYTSGTTGDPKGVLISNASIICLVAGVDRLLNCVNEQLEQTDVYMSYLPLAHIFDRVVEELFMFHGASIGFWRGDVKLLVEDIGTLKPTILCAVPRVLDRIFSGLQAKIASGGFIKSTLFNLAYKFKQFRMMKGAKHNEAAAICDKVVFSKVKEGLGGNVRVILSGAAPLATHVEEYLRVVTCAHVLQGYGLTETCAGSFVSLPNQMCMIGTVGPPVPNIDVCLESVPEMNYDALATRPRGEICIRGETVFSGYYKREDLTKDVLIDGWFHTGDIGEWQPDGSMKIIDRKKNIFKLSQGEYVAVENLENIYGLVSAIDSIWVYGNSFESFLVAVVNPNKEALESWAAANGISGDLEALCENPKAKEYILGELSKVGKEKKLKGFEFIKAVHLEPVPFDMDRDLITPTYKKKRPQLLKYYQGTIDNMYRSAK

SEQ ID NO: 15 - protein, 532 aa, Oryza sativa
MEVGTWAVVVAVAAAYMTWFWRMSRGLSGPRVWPVVGSLPGLVQHAENMHEWIAANLRRAGGTYQTCIFAVPGVARRGGLVTVTCDPRNLEHVLKSRFDNYPKGPFWHAVFRDLLGDGIFNSDGETWVAQRKTAALEFTTRTLRTAMSRWVSRSIHHRLLPILDDAAAGKAHVDLQDLLLRLTFDNICGLAFGKDPETLAKGLPENAFASAFDRATEATLNRFIFPEYLWRCKKWLGLGMETTLASSVAHVDQYLAAVIKARKLELAGNGKCDTVAMHDDLLSRFMRKGSYSDESLQHVALNFILAGRDTSSVALSWFFWLVSTHPAVERKVVHELCAVLAASRGAHDPALWLAAPFTFEELDSLVYLKAALSETLRLYPSVPEDSKHVVADDYLPDGTFVPAGSSVTYSIYSAGRMKTVWGEDCLEFRPERWLSADGSKFEPHDSYKFVAFNAGPRICLGKDLAYLQMKNIAGSVLLRHRLAVAQGHRVEQKMSLTLFMKNGLRMEVRPRDLAPVADELRGADVRATAPCA

SEQ ID NO: 16 - protein, 545 aa, Oryza sativa
MEAGTWAVVVAAVAAYMAWFWRMSRGLSGPRVWPVVGSLPGLVRHAEDMHEWIAANLRRTRGTYQTCIFAVPGLARRGGLVTVTCDPRNLEHVLKSRFDNYPKGPFWHGVFGDLLGDGIFNSDGETWVAQRKTAALEFTTRTLRTAMSRWVSRSIHSRLLPILSDAAAAGGGGGGATVDLQDLLLRLTFDNICGLAFGKDPETLARGLPENDFASAFDRATEATLNRFIFPECVWRFKKWMGLGMETTLARSVQHVDRYLSAVIKARKLELAAGNGKGDASSATPHDDLLSRFMRKGTYSDESLQHVALNFILAGRDTSSVALSWFFWLVSTHPAVERKIVRELCTVLAASRGADDPALWLAAPLNFEELDQLVYLKAALSETLRLYPSVPEDSKHVVADDVLPDGTFVPAGSSVTYSIYSAGRMKTVWGDDCLEFRPERWLSADGTKFEPHDSFRFVAFNAGPRICLGKDLAYLQMRNIAGSVLLRHRLAVAPGHRVEQKMSLTLFMKHGLRMEVRPRDLAPIVDELRGAGEYAAAARATAACA

SEQ ID NO: 17 - protein, 509 aa, Oryza sativa
MMASDRLDAERIIEYFKGKSILITGATGFLGKILVEKILRVQPDVKKIYLLVRAIDQASANQRVQSEVTETELFSVVKEKHGKGFSRFIEEKVVALAGDIIYDDLGLDPPLLQHLADNLDVIVNGAATTNFYGRYDVSLDVNVLGVKHLCQLAKKCRGLKVFLHVSTAYAGGEQEGLIQERAFEEGWALREGMHLDVDAELRLVADVRREVEDDDDKARRKAMKELGLARARHFGWSNTYVFTKAMGEMVLSRMLLQSSSPAPAITGAGGVVVVRPSIITSIQRDPVPGWMQGTRTIDTLIIGYAKQNLSCFLGDLDLVMDVIPGDMVVNAMMAAAVAHSGEAGQERPAVYHVSSSLRNPAAYSVLYEAGRRHFTEKPRVGKRGEVIPTKEMHFFKTIASFQVYMLVKYRLPLEILHLVNLLLCGLFSRLYSNLARQYRYVMHLVDVYGPFAFFKGCFDDINLERLRQRMGKSRNPQDDEMFNFDPKTIDWEDYFYRIHIPGVLKYILK

SEQ ID NO: 18 - protein, 505 aa, Oryza sativa
MVGTMDEARIIEYFKNKSILITGSTGFLGKILVEKILRVQPDVRKIYLPVRAVDAATAKQRMQTEVIGKELFGLLKEQHGKGFQSFIDEKVVPLAADMMHQNLGLEESTLQELAKDLNIIVNGAATTNFYERYDVALDVNVMGVKYLCQLAKKCANLEVFLHVSTAYVCGERSGVVQERALREGETLREGTYLDIETELRLVGEQRQQLEDAGDAKAERKAMKDLGLARARHFGWPNTYVFTKAMGEMMLQEQLVAGAGRRHGIPVVIARPSIITSVHRDPLPGWIEGTRTIDAIIIGYAKQSLSCFLADLDLIMDVVPGDLVVNAMMAAMVAHSRGSSSEMAVYHVTSSMRHPAAYAVLYRTGWRYFLENPRVGKDGVAVRTRPVYFFRTIASFRAFMAVAYALPLQLLRLLSLLCFGLLFARRYADLSRKYSFVMQLVDLYGPFALFKACFDDLNMEKLRLSMATPPSSAAAALFNLDPKNIDWDDYFYRIHIPGVMKYVLNK

SEQ ID NO: 19 - protein, 494 aa, Oryza sativa
MKLLYHHAISNAPYLMLAAAAAAVALRASRLAPADDLATARELLATNLPLAVALLAAAVVLATAYLMRRPRPVYLLDFACYKPGPEHVVTRETFMAQSAAAGAFTGDSLAFQRKILERSGLGQGTYFPAAVLNSPPNPCMAEARREAEQVMFGAIDAVLAKTGVRARDIGVVVVNCSLFNPTPSLSAMIVNHYKLRGNVATYNLGGMGCSAGLISIDLAKQLLQVHRNSYALVVSMENITLNWYWGNNRSMLVSNCLFRMGGAAILLSNRGGDRRRAKYQLLHTVRTHSGAADRAYRCVFQEEDDAAGVGVALSKDLMAVAGEALRTNITTLGPLVLPMSEQILFLASLVARRVFGLAGVRPYIPDFKMAFDHFCIHAGGRAVLDTIEKNLGLGAWHMEPSRMTLYRWGNTSSSSLWYELAYAEAKGRVRRGQTAWQIAFGSGFKCNSAVWRALRTVEPDADERNPWAGEIDSFPVEVPKVEAVATATADAASS

SEQ ID NO: 20 - protein, 340 aa, Oryza sativa
MGRSPCCCHDAGVKKGPWTEEEDRALVEHIKKQGGHVGSWRGLPRAAGLNRCGKSCRLRWTNYLRPDIRRGNFSDDEERLIIRLHAALGNKWSTIATHLDGRTDNEIKNYWNTHIKKKLLRMGIDPVTHQRLPPDLLADGGGLGAASPLLSPPGPAAAAALQPLLSAVASLGSLDTALRQFQLLQHLLNSITSSSSDVAATAGLMATNLAATNTMVNSSSNVASFQEQMNALAHANYQPGYLRDVVPSFPGQDMAPQLNSTSSTPSTAPVLRSSAEPADQCCNDAALVPETYPREVAASVDHWKVQDFPSLEPLELPNLSTLESDLDPFWKEILESSFRS

SEQ ID NO: 21 - DNA, 30 bp, synthetic construct
  source 1..30: mol_type = other DNA
cccaaaaagaagaggaaagttggcgggtga